(19) United States
(12) Patent Application Publication
(10) Pub. No.: US 2021/0186993 A1
(43) Pub. Date: Jun. 24, 2021

(54) COMBINATION THERAPIES TARGETING MITOCHONDRIA FOR CANCER THERAPY

(71) Applicant: The Wistar Institute of Anatomy and Biology, Philadelphia, PA (US)

(72) Inventors: Dario C. Altieri, Philadelphia, PA (US); Jagadish C. Ghosh, Aldan, PA (US)

(21) Appl. No.: 16/987,974

(22) Filed: Aug. 7, 2020

Related U.S. Application Data
(63) Continuation of application No. 16/254,919, filed on Jan. 23, 2019, now abandoned.
Application No. 16/254,919 is a continuation of application No. 15/308,130, filed on Nov. 1, 2016, now abandoned.
(60) Provisional application No. 61/987,720, filed on May 2, 2014.

(51) Int. Cl. A61K 31/665; A61K 45/06; A61K 31/519; A61K 31/66; A61K 31/4745; A61K 31/501; A61K 31/5377; A61K 31/585

(52) U.S. Cl. CPC A61K 31/665; A61K 45/06; A61K 31/519; A61K 31/585; A61K 31/4745; A61K 31/501; A61K 31/5377; A61K 31/66

(57) ABSTRACT

Pharmaceutical compositions for the treatment of cancer are provided. In one embodiment, the composition comprises Gamitrinib and a PI3K inhibitor selected from PX-866, AZD6482, LY294002, BEZ235, GSK458, GDC0941, ZSTK474, BKM120 and GSK2636771B. Methods of treating cancer are also provided.

### CROSS-REFERENCE TO RELATED APPLICATIONS

This application is continuation of U.S. patent application Ser. No. 16/254,919, filed Jan. 23, 2019, which is a continuation of U.S. patent application Ser. No. 15/308,130, filed Nov. 1, 2016, which is a national stage of International Patent Application No. PCT/US2015/028850, filed May 1, 2015, which claims the benefit of the priority of U.S. Provisional Patent Application No. 61/987,720, filed May 2, 2014, which applications are incorporated by reference herein in their entireties.

### STATEMENT OF GOVERNMENT INTEREST

This invention was made with government support under grant numbers CA140043 and CA78810 awarded by the National Institutes of Health. The government has certain rights in the invention.

### BACKGROUND

Adaptive mechanisms buffer environmental stress during tumor ontogeny, and may create new cancer phenotypes. Such tumor plasticity is important for disease progression because it promotes resistance to therapy, dormancy and acquisition of metastatic propensity, but its effectors are largely unknown.

The phosphoinositide 3-kinases (PI3Ks) are a family of lipid kinases that involved in intracellular signaling, The PI3K pathway is a universal signaling node that integrates environmental cues of cellular growth with downstream networks of cell proliferation, survival, and bioenergetics. Exploited in virtually every human cancer, in some cases through the acquisition of activating mutations, PI3K signaling and its effectors Akt and mammalian target of rapamycin (MTOR) are validated therapeutic targets, and several small molecule antagonists of this pathway have entered clinical testing.

PI3K phosphorylates the 3′-OH group on phosphatidylinositols in the plasma membrane. This leads to recruitment and activation of the protein Ser/Thr-kinase. AKT, to the cell membrane, The PI3K/AKT signaling cascade is critical in cancer as it promotes cell survival and growth. PI3K-AKT signaling is activated in cancers by several different mechanisms, and inhibitors of the PI3K pathway are in development as cancer therapies.

However, the response to PI3K therapy in the clinic has been inferior to expectations, with modest single-agent activity, statistically significant toxicity, and short-lived patient benefits. The basis for this treatment resistance is unknown, and strategies to guide patient selection or incorporate PI3K therapy in more effective combination regimens have remained elusive. In this context, there is evidence that small-molecule inhibitors of PI3K/Akt/MTOR activate a broad transcriptional and signaling program in tumors, culminating with a paradoxical (re)activation of Akt in treated patients. How (and whether) this process contributes to drug resistance has not been clearly elucidated, but it is possible that it provides a general adaptive response to “environmental stress” imposed by molecular therapy. In this context, mechanisms of adaptation are important drivers of tumor diversity and treatment failure, hinging on a tight control of the protein-folding environment by molecular chaperones of the Heat Shock Protein-90 (Hsp90) family.

Heat Shock Protein-90 (Hsp90) chaperones oversee protein folding quality control in every organism. This process is essential for cellular homeostasis, buffering proteotoxic stress, and enabling cells to continuously adapt to changes in their internal and external milieus. Hsp90 plasticity has been traditionally linked to the diversity of its ‘client proteins’, molecules implicated in multiple facets of cellular maintenance and that require the chaperone ATPase activity for proper folding, maturation, and subcellular trafficking. However, successful adaptation must also encompass fine-tuning of bioenergetics, nutrient-sensing and stress response signaling networks, including autophagy, and the role of Hsp90 in these pathways has remained unexplored.

### SUMMARY

The present invention is based in part on the inventor's discovery that combination therapy that targets mitochondria eliminates tumor adaptation induced by PI3K inhibition, and improves clinical outcome in cancer.

In one aspect is provided a pharmaceutical composition comprising gamitrinib and a PI3K inhibitor. In one embodiment the composition comprises gamitrinib and PX-866. In another embodiment the composition comprises gamitrinib and AZD6482. In one embodiment the composition comprises gamitrinib and LY294002. In one embodiment the composition comprises gamitrinib and BEZ235. In another embodiment the composition comprises gamitrinib and GSK458. In one embodiment the composition comprises gamitrinib and GDC0941. In yet another embodiment the composition comprises gamitrinib and ZSTK474. In one embodiment the composition comprises gamitrinib and BKM120. In yet another embodiment, the composition comprises gamitrinib and GSK2636771. In yet another embodiment, the composition comprises gamitrinib and GDC0980.

In another aspect a method of treating cancer in a subject in need thereof is provided. The method includes administering to the subject a pharmaceutically effective amount of a composition comprising gamitrinib and a PI3K inhibitor, as described herein.

Further aspects will be readily apparent based on the description provided herein.

### BRIEF DESCRIPTION OF THE DRAWINGS

FIGS. 1A-1J demonstrate metabolic and transcriptional tumor reprogramming induced by PI3K inhibition. a-c. Modulation of oxidative phosphorylation (a), fatty acid β oxidation (b), and polyamine metabolism (c) in LN229 cells treated with PX-866 (10 μM for 48 h) (n=5). Red, up-regulation; green, down-regulation. d-g. Changes in ATP production (d), glucose utilization (e), lactate generation (f), or oxygen consumption (g) in tumor cells treated with vehicle (Veh) or PX-866 (10 μM for 48 h). Mean±ESEM. **, p=0.007; ***, p<0.0001. h-i. Heatmaps of changes in kinome pathways (h) or functions (i) in glioblastoma (GBM) organoids treated with vehicle or PX-866, alone or in combination with Gamitrinib (5 μM, see also FIG. 4). N=number of genes; N_{G}/N_{G} %=number/percent of genes that show reduced PX-866 effect (>2 fold) by Gamitrinib; %=percent of genes upregulated in the pathway; Z=z-score. Positive (red)=increased; negative (blue)=decreased. j. Phospho-RTK array of vehicle- or PX-866-treated (IBM organoids. M, markers. See also FIG. 17.

FIGS. 2A-2E demonstrate tumor plasticity induced by PI3K inhibition. a-b. Treated tumor cells were stained for senescence-associated-β-galactosidase (SA-β-Gal) and quantified (a) or PML nuclear body number (b) and quantified, Mean±SEM. *, p=0.01; **, p=0.006-0.007; *** p=0.0002. c-d. Treated tumor cells were analyzed for invasion across Matrigel-coated Transwell membranes (c) or from 3D spheroids embedded in a collagen matrix as the distance between core (red contour) and invasive edge (green contour) (d), and quantified (right panels). Mean±SEM. *, p=0.02***, p<0.0001. e. Treated tumor cells were analyzed by Western blotting. p, phosphorylated.

FIGS. 3A-3M demonstrate Akt regulation of tumor plasticity. a. Ser473-phosphorylated Akt (pAkt) was analyzed in GBM organoids treated with vehicle (Veh) or PI3K inhibitors, LY294002 (50 μM) or PX-866 (10 μM) by immunofluorescence (data not shown), and quantified (a) (LY294002, 50-100 μM; PX-866, 2.5, 5, 10 μM, 17-AAG, 20 μM ). None, untreated. *, p=0.02-0.03; **, p=0.001.-0.003, ***, p<0.0001. b. Mitochondrial (Mito) or cytosolic (Cyto) fractions from treated LN229 cells were analyzed by Western blotting. c. Analysis of mitochondrial (Mito) or cytosolic (Cyto) fractions from prostate tissues of wild type (Pten^{+/+}) or Pten^{pc-/-} mice (3 mice per condition). *, non-specific. d-e. Recombinant wild type (WT) (d) or mutant (e) CypD proteins were incubated with vehicle (Veh) or active Akt2 in a kinase assay with ³²P-ATP followed by autoradiography. GSK3β was used as a control Akt substrate. f CypD^{-/-} MEFs reconstituted with WT or mutant CypD were analyzed for peptidyl prolyl cis,trans isomerase (PPIase) activity. CypD H168Q isomerase-defective mutant was a control. g-j. LN229 cells with stable shRNA knockdown of CypD were reconstituted with CypD cDNAs and analyzed by Western blotting (g), or changes in activity (h), glucose utilization (i) or oxygen consumption (j). Mean±SEM. *, p=0.042-0.01, **, p=0.0012-0.0014. k-m, Reconstituted LN229 CypD knockdown cells or CypD^{-/-} MEFs were analyzed for mitochondrial membrane potential (k), or Annexin V labeling (l) by multiparametric flow cytometry, or loss of cell viability by MTT (m). For panels m and n, the percentage of cells in each quadrant is indicated. Mean±SEM. ***, p<0.0001.

FIGS. 4A-4E demonstrate the requirement of mitochondrial function for tumor plasticity. a-b. Nude mice injected with U87-Luc glioblastoma cells in the right cerebral striatum were treated as indicated, and analyzed by bioluminescence imaging (data not shown), with signal quantification in the indicated groups 28 days after injection (a). b. Overall survival. **, p=0.0015; *, p=0.01-0.04. c. Breast cancer organoids treated with PX-866 (10 μM) alone or in combination with Gamitrinib (10 μM) were analyzed by immunohistocheinistry and fluorescence microscopy (data not shown), and pAkt-expressing cells were quantified (c). d. Heatmaps of Reverse Phase Protein Array with significant changes in protein expression and/or phosphorylation in tumor cells treated with PX-866 or Gamitrinib, alone or in combination. N=3. e. Breast adenocarcinoma organotypic cultures treated with PX-866 (10 μM) alone (left) or in combination with Gamitrinib (10 μM, right) and pAkt- or pMTOR-expressing cells were quantified.

FIGS. 5A-5D demonstrate metabolic reprogramming induced by PI3K inhibition. LN229 cells were treated with vehicle (left bars) or 10 μM, PX-866 (right bars) and analyzed for changes in the expression of the indicated metabolites after 48 h (n=5, p<0.05). For box plots the limit of upper and lower quartiles, median values (straight line), and maximum and minimum of distribution are indicated. Cross, mean value; circle, extreme data point, a, oxidative phosphorylation; b, long-chain fatty acids; c, carnitine conjugates in β-oxidation; d, polyamine metabolism.

FIGS. 6A-6B demonstrate transcriptional reprogramming induced by PI3K inhibition. a. RNA extracted from vehicle- or PX-866-treated GBM organotypic cultures was amplified for the indicated gene products, by quantitative PCR. b. Extracts from LN229 cells treated with vehicle (Veh) or PX-866 (10 μM for 48 h) were hybridized with a human phospho-RTK array, and immunoreactive spots were identified by autoradiography. The position and identity of modulated RTKs are indicated. M, markers.

FIGS. 7A-7D demonstrate tumor phenotypes induced by PI3K inhibition. LN229 (a) or PC3 (b) cells were treated with vehicle or PX-866 (10 μM) and analyzed for DNA content by PI staining and flow cytometry after the indicated time intervals. The percentage of cells in each cell cycle transition is indicated. c. The indicated cell types were treated with vehicle (Veh) or PX-866 (10 μM) and analyzed by direct cell counting at the indicated time intervals. d. LN229 cells were treated with the indicated concentrations of PX-866 and analyzed for cell viability after 48 h by Trypan blue exclusion (left) or an MTT assay (right) relative to vehicle-treated cultures.

FIGS. 8A-8C demonstrate reactivation of Akt signaling after PI3K inhibition, in vivo. a-b. Organotypic cultures of representative cases of infiltrating ductal breast adenocarcinoma (Breast AdCa, a) or colon adenocarcinoma (b) were treated with vehicle (Veh) or the indicated concentrations of pan-PI3K, inhibitor, LY294002 (LY) and analyzed for changes in Ser473-phosphorylated Akt (pAkt) after 48 h, by fluorescence microscopy (data not shown). DNA was stained with DAPI. Cytokeratin was an epithelial marker. Quantification of pAkt⁺ cells in the indicated tumor organoid. None, untreated. *, p=0.02; ***, p=0.0008. b. LN229 cells were treated with 10 μM PX-866 and analyzed at the indicated time intervals by Western blotting. c. Tumor cell lines were treated with the indicated increasing concentrations of PX-866 and analyzed by Western blotting. See also FIGS. 18 and 19.

FIGS. 9A-9H demonstrate characterization of mitochondrial Akt. a. LN229 cells were treated with vehicle (Veh) or LY294002 (LY, 50 μM for 48 h), fractionated in cytosol (Cyto) or mitochondrial (Mito) extracts and analyzed by Western blotting, b. LN229 cells were fractionated in cytosolic (C) or mitochondrial (M) extracts and analyzed by Western blotting. c. Sub-mitochondrial fractions isolated from LN229 cells comprising outer membrane (OM), inter-membrane space (IMS), inner membrane (IM) or matrix were analyzed by Western blotting. TME, total mitochondrial extracts. d. Mitochondria isolated from LN229 cells (Mito) were treated with the indicated increasing concentrations of proteinase K and analyzed by Western blotting. Proteinase K-dependent proteolysis in the cytosol (Cyto) is shown as control. COX-IV was used as a mitochondrial marker protected from proteolysis. Bcl-2 was used a mitochondrial outer membrane-localized protein susceptible to proteinase K proteolysis. e-f. The indicated tumor or normal (MRC5) cell lines (e) or primary mouse tissues (f) were fractionated in cytosolic (Cyto) or mitochondrial (Mito) extracts and analyzed by Western blotting. g. LN229 cells were incubated in normoxia (N) or hypoxia (H, 0.5% O₂) conditions for 24-48 h, fractionated in cytosol (Cyto) or mitochondrial (Mito) extracts and analyzed by Western blotting. TCE, total cell extracts.HK-II was used as a control for a hypoxia-regulated mitochondrial imported protein. h. LN229 cells were treated with 200 mM H₂O₂, 200 nM Thapsigargin (Thaps) or 20 mM 2-deoxyglucose (2-DG) for 24 h, and isolated cytosolic (Cyto) or mitochondrial (Mito) extracts were analyzed by Western blotting. TCE, total cell extracts.

FIGS. 10A-10J demonstrate mitochondrial Akt2 phospholation of CypD. a. Mitochondrial extracts from LN229 cells were immunoprecipitated (IP) with IgG or an antibody to CypD and pellets or supernatants (Sup) were analyzed by Western blotting. IgG_{L}, immunoglobulin light chain. b. Recombinant GST-CypD or GST was incubated with mitochondrial extracts of LN229 cells and bound proteins were analyzed by Western blotting. CB, Coomassie Blue staining. c. Recombinant GST-CypD or GST was incubated with ³⁵S-labeled Akt, and bound proteins were visualized by autoradiography. d. CypD sequence analysis. Potential Akt phosphorylation sites are indicated in red. e. The indicated recombinant proteins were incubated with active Akt in a kinase assay and radioactive proteins were visualized by autoradiography. GSK3β was used as a control Akt substrate. f. LN229 cells with stable shRNA knockdown of CypD were transfected with vector, wild type (WT) or Ser31→Ala (S31A) CypD cDNA, immunoprecipitated (IP) with IgG or an antibody to CypD and pellets were analyzed by Western blotting. P-Ser, phosphorylated. Ser. g. WT or CypD^{-/-} MEFs were transfected with the indicated constructs and analyzed by Western blotting. h-j. LN229 cells with stable shRNA knockdown of CypD were transfected with the indicated plasmid constructs and analyzed by Western blotting (h,j) or changes in ATP production (i). Mean±SEM. *, p=0.026; **, p=0.004. See also, FIG. 23.

FIGS. 11A-11E demonstrate high throughput screening of Gamitrinib-PI3K inhibition combination, a.-c. LN229 cells were incubated with increasing concentrations of anti-cancer agents in the presence of vehicle (DMSO) or Gamitrinib (1 μM) and analyzed for changes in cell viability after 72 h. See Table 2. The heatmap represents compounds (n=48) with significant increased inhibitory effect (p<0.05) in the presence of 1 μM Gamitrinib. Conc.=concentration; p=p-value (Wilcoxon test); avg=average difference between inhibition shown in Gamitrinib vs. DMSO across all concentration points, 2=results from the combination of targeted compounds plus 2 μM Gamitrinib versus DMSO (only a subset of compounds were tested). d. LN229 cells were incubated with the indicated increasing concentrations of small molecule inhibitors of PI3K (GSK458, BKM120, BEZ235, GDC0941, AZD6482) or Akt (MK2206) in the presence of vehicle (Veh) or Gamitrinib (1 μM) and analyzed for changes in cell viability after 72 h. Mean±SEM. e. U251 cells were treated with NVP-BEZ235 (BEZ. 0.5 μM) or LY294002 (LY, 50 μM) alone or in combination with Gamitrinib (0.5 μM or 5 μM, respectively), and analyzed for mitochondrial membrane potential by JC1 staining (top) or Annexin V labeling (bottom), by multiparametric flow cytometry. The percentage of cells in each quadrant is indicated.

FIGS. 12A-12E demonstrate Gamitrinib-PI3K inhibition synergy, a-b. U251 cells were treated with LY294002 (LY, 50 μM) or Gamitrinib (5 μM), alone or in combination and analyzed by Western blotting. c-d. U251 cells were transfected with vector or Δ-p85 PI3K subunit, alone or in combination with Gamitrinib, and analyzed by Western blotting (c) or cell viability by MTT (d). Mean±SEM. ***, p=0.0002. e. LN229 cells were treated with increasing concentrations of Gamitrinib (2-0.008 μmol/L) in combination with MK kinase inhibitors, NVP-BEZ235 (BEZ), GDC-0941 or AZD6482 (2-0.008 μmol/L) in a 7×7 matrix for 18 h at 37° C. Cell viability was measured by addition of resazurin, and the fractional growth inhibition was calculated. Left, Excess over Bliss; Right, % growth inhibition.

FIGS. 13A-13C demonstrate anticancer activity of Gamitrinib-PI3K inhibition combination, A-b. Nude mice were injected with U87-Luc cells in the right cerebral striatum, treated with vehicle (Veh), NVP-BEZ235 (BEZ), or Gamitrinib (Gam) alone or in combination, and analyzed for luciferase expression by bioluminescence imaging at the indicated time intervals after injection. Tissue sections from U87-Luc gliomas in the various groups were analyzed for Ki-67 expression (a) or apoptosis by TUNEL staining (b). The percentage of positive cells is indicated. **, p=0.0065;*** p<0.0001. c. Heatmap of Reverse Phase Protein Array (RPPA) with significant changes in protein expression and/or phosphorylation in LN229 cells treated with PX-866 or Gamitrinib alone or in combination. N=3.

FIG. 14 are graphical representations showing the percent inhibition vs. log of increasing concentrations of DMSO (control) or Gamitrinib (Gami) in combination with PI3K inhibitors BKM120, GSK458, BEX235, GDC0941, and AZD6482.

FIGS. 15A-15C demonstrate the effect of PI3K therapy on mitochondrial metabolic reprogramming. LN229 or PC3 cells were treated with vehicle or PI3K inhibitors, PX-866 (10 μM), AZD6482 (10 μM), or GDC0942 (2 nM) and analyzed after 48 hours for changes in glucose utilization (A), lactate generation (B), or ATP production (C), Mean±SD of at least two independent determinations. MTA=5′-deoxy-5′-(methylthio)adenosine; Veh=vehicle. See also, FIG. 1.

FIGS. 16A-16C demonstrate PI3K therapy on regulation of Akt signaling. A) The percentage of pAkt+ cells or a pMTOR immunohistochemical score was quantified in GBM organotypic cultures (PX-866, 2.5, 5, 10 μM, 17-AAG, 20 μM). None=untreated. Mean±SD of at least three independent determinations. *P=0.03-0.04; ** P=0.005 by two-sided unpaired t test. B) The various tumor cell lines were treated with the indicated increasing concentrations of PX-866 and analyzed by western blotting. C) LN229 cells were treated with vehicle or LY294002 (LY, 50 μM for 48 hours), fractionated in cytosol or mitochondrial extracts, and analyzed by western blotting. C) LN229 cells were incubated in normoxia or hypoxia (H, 0.5% O2) conditions for 24 to 48 hours, fractionated in cytosol or mitochondrial extracts and analyzed by western blotting. HK-II was used as a control for a hypoxia-regulated mitochondrial-associated protein. Cyto=cytosol; GBM=glioblastoma; Mito=mitochondrial; N=normoxia; NSE=neuron-specific enolase; pAkt=Ser473-phosphorylated Akt; TCE=total cell extracts; Veh=vehicle.

FIG. 17 is a western blot of LN229 cells with stable shRNA knockdown of CypD were transfected with the indicated FLAG-tagged. CypD cDNAs, treated with vehicle or PX-866, and immunoprecipitated with anti-FLAG M2 gel followed by western blotting with anti-pSer antibody. The position of full-length or mature CypD band is shown.

FIGS. 18A-18B demonstrates the effect of PI3K therapy on tumor cell proliferation and survival. A and B) LN229 (A) or PC3 (B) cells were treated with vehicle (Veh) or PI3K inhibitors PX-866 (10 μM), AZD6482 (10 μM) or GDC0941 (2 μM) and analyzed for DNA content by propidium iodide staining and flow cytometry after 24 and 48 h. The percentage of cells in the various cell cycle phases is indicated. See also FIG. 8 and FIG. 19.

FIGS. 19A-19E demonstrate the relationship of Akt signaling and PI3K therapy. A) Organotypic cultures of GBN were treated with vehicle (Veh) or the indicated concentrations (μM) of pan-PI3K inhibitor, LY294002 (LY, 50-100 μM) and analyzed for changes in Ser473-phosphorylated Akt (pAkt) after 48 h, by fluorescence microscopy (data not shown) and quantified. DNA was counterstained with DAPI. None, untreated. NSE, neuron-specific enolase, Cytokeratin (CK) was an epithelial marker. Scale bar=100 (B) The percentage of pAkt⁺ cells or a pMTOR immunohistochemical score was quantified in colon AdCa tumor organotypic cultures treated with LY294002 (μM), *, p=0.02 by two-sided unpaired t test. None, untreated. All data are expressed as mean±SD of individual replicates. C) PC3 cells were transfected with control non-targeting siRNA (Ctrl) or siRNA directed to PI3K p110α subunit, and analyzed in two independent experiments after 72 h, by Western blotting. p, phosphorylated. D) The indicated melanoma cell lines were treated with the indicated increasing concentrations of PX-866 and analyzed by Western blotting. The BRAF genotype is indicated per each cell line tested. E) PC3 cells were treated with vehicle (Veh) or PI3K inhibitors AZD6482 (10 μM), GDC0942 (0.4-2 μM) or BKM120 (0.4-2 μM) and analyzed after 48 h by Western blotting.

FIG. 20A-20C demonstrate mitochondrial Akt. LN229 (top)or PC3 (bottom) cells were fractionated in cytosolic (C) or mitochondrial (M) extracts and analyzed with isoform-specific antibodies to Akt(Akt1, Akt2 or Akt3), by Western blotting. B) LN229 cells were treated with the indicated increasing concentrations of PX-866 for 48 h, fractionated in cytosol (Cyto) or mitochondrial (Mito) extracts and analyzed by Western blotting. C) LN229 cells were treated with vehicle (Veh), PX-866 (10 μM), or 17-AAG (1 μM), alone or in combination for 48 h, fractionated in cytosol (Cyto) or mitochondrial (Mito) extracts, and analyzed by Western blotting.

FIG. 21 demonstrates mitochondrial Akt2 phosphorylation of CypD. Wild type (WT) or LN229 cells with stable shRNA knockdown of CypD were transfected with the indicated cDNAs in the presence of vehicle (Veh) or PX-866 (10 μM for 48 h), and analyzed by Western blotting. The experiment was repeated at least twice with comparable results.

### DETAILED DESCRIPTION OF THE INVENTION

The compositions and methods provided herein are based on the inventors' discovery that combination therapies that target mitochondria eliminate tumor adaptation induced by PI3K inhibition. As discussed herein, the inventors have shown that perturbation of the phosphatidylinositol-3 kinase (PI3K) pathway, a disease driver in virtually every cancer and validated therapeutic target, induces global metabolic and transcriptional reprogramming in tumors. This creates a new cancer phenotype that combines paradoxical traits of bioenergetics and cellular quiescence, heightened cell survival and increased tumor cell invasion. Its underpinning is a (re)activation of Akt in multiple subcellular compartments, including mitochondria, with phosphorylation-dependent repurposing of mitochondrial functions. Conversely, disabling mitochondrial quality control reverses tumor reprogramming induced by PI3K therapy, and potently enhances anticancer activity. As demonstrated in the examples below, molecular therapies are powerful drivers of tumor plasticity and more aggressive behavior, whereas combination therapy that targets mitochondria eliminates tumor adaptation and improves clinical responses in cancer patients.

### I. Definitions

Technical and scientific terms used herein have the same meaning as commonly understood by one of ordinary skill in the art to which this invention belongs and by reference to published texts, which provide one skilled in the art with a general guide to many of the terms used in the present application. The following definitions are provided for clarity only and are not intended to limit the claimed invention.

The terms “a” or “an” refers to one or more, for example, “a Gamitrinib” is understood to represent one or more Gamitrinib compounds. As such, the teens “a” (or “an”), “one or more,” and “at least one” are used interchangeably herein. As used herein, the term “about” means a variability of 10% from the reference given, unless otherwise specified. While various embodiments in the specification are presented using “comprising” language, under other circumstances, a related embodiment is also intended to be interpreted and described using “consisting of” or “consisting essentially of” language.

“Patient” or “subject” as used herein means a mammalian animal, including a human, a veterinary or farm animal, a domestic animal or pet (including cats and dogs), and animals normally used for clinical research (including mice, rats, non-human primates. etc). In one embodiment, the subject of these methods and compositions is a human.

The term “cancer” or “proliferative disease” as used herein means any disease, condition, trait, genotype or phenotype characterized by unregulated cell growth or replication as is known in the art. A “cancer cell” is cell that divides and reproduces abnormally with uncontrolled growth. This cell can break away from the site of its origin (e.g., a tumor) and travel to other parts of the body and set up another site (e.g., another tumor), in a process referred to as metastasis. A “tumor” is an abnormal mass of tissue that results from excessive cell division that is uncontrolled and progressive, and is also referred to as a neoplasm. Tumors can be either benign (not cancerous) or malignant. The methods described herein are useful for the treatment of cancer and tumor cells, i.e., both malignant and benign tumors, so long as the cells to be treated have mitochondrial localization of the chaperones as described herein. In various embodiments of the methods and compositions described herein, the cancer can include, without limitation, breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, acute and chronic lymphocytic and myelocytic leukemia, myeloma, Hodgkin's and non-Hodgkin's lymphoma, and multidrug resistant cancer. In one embodiment, the cancer is a drug resistant cancer.

As used herein, the term “any intervening amount”, when referring to a range includes any number included within the range of values, including the endpoints.

The term “regulation” or variations thereof as used herein refers to the ability of a compound of a compound or composition described herein to inhibit one or more components of a biological pathway.

As used herein, “disease”, “disorder” and “condition” are used interchangeably, to indicate an abnormal state in a subject. The term “treating” or “treatment” is meant to encompass administering to a subject a compound described herein for the purposes of amelioration of one or more symptoms of a disease or disorder.

### II. Compositions

In one aspect, pharmaceutical compositions are provided. In one aspect, the pharmaceutical composition comprises gamitrinib and a PI3K inhibitor. In one embodiment, the PI3K inhibitor is PX-866. In another embodiment, the PI3K inhibitor is AZD6482. In another embodiment, the PI3K inhibitor is LY294002. In another embodiment, the PI3K inhibitor is BEZ235. In another embodiment, the PI3K inhibitor is GSK458. In another embodiment, the PI3K inhibitor is GDC0941. In another embodiment, the PI3K inhibitor is ZSTK474. In another embodiment, the PI3K inhibitor is BKM120. In another embodiment, the PI3K inhibitor is GSK2636771. In another embodiment, the PI3K. inhibitor is GSK458, In another embodiment, the PI3K inhibitor is GDC-0980.

Gamitrinib is a molecule that inhibits selectively the pool of Hsp90 localized to mitochondria of tumor cells. As used herein, the term “Gamitrinib” refers to any one of a class of geldanamycin (GA)-derived mitochondrial matrix inhibitors. Gamintrinibs contain a benzoquinone ansamycin backbone derived from the Hsp90 inhibitor 17-(allylamino)-17-demethoxygeldanamycin (17-AAG), a linker region on the C17 position, and a mitochondrial targeting moiety, either provided by 1 to 4 tandem repeats of cyclic guanidinium (for example, a tetraguanidinium (G4), triguanidinium (G3), diguanidinium (G2), monoguanidinium (G-1),) or triphenylphosphonium moiety (Gamitrinib-TPP-OH). For example, Gamitrinib-G4 refers to a Gamitrinib in which a tetraguanidinium moiety is present. For example, Gamitrinib-TPP refers to a Gamitrinib in which a triphenylphosphonium moiety is present. Also throughout this application, the use of the plural form “Gamitrinibs” indicates one or more of the following: Gamitrinib-G4, Gamitrinib-G3, Gamitrinib-G2, Gamitrinib-G1, and Gamitrinib-TPP or Gamitrinib-TPP-OH. Gamitrinib is a small molecule inhibitor of Hsp90 and TRAP-1 ATPase activity, engineered to selectively accumulate in mitochondria. In one embodiment, the Gamintrinib is Gamitrinib-TPP-OH. In another embodiment, the Gamitrinib is Gamitrinib-G4. The approximate molecular weights of the Gamitrinibs discussed herein are the following: Gamitrinib-G1: 1221.61 g/mol; Gamitrinib-G2: 709.85 g/mol; Gamitrinib-G3: 539.27 g/mol; Gamitrinib-G4: 604.97 g/mol; and Gamitrinib-TPP: 890.46 g/mol. See, e.g., United States Patent Publication No. 2009/0099080 and Kang et al, 2009, J. Clin. Invest, 119(3):454-64 (including supplemental material), which are hereby incorporated by reference in their entirety.

The terms “mitochondria-penetrating moiety” and “mitochondria-targeting moiety” are used herein interchangeably. In one embodiment, by “mitochondria-penetrating moiety” or “mitochondria-targeting moiety” it is meant a molecule that targets to and, together with its cargo, accumulates in mitochondria due to its: i) high affinity binding to one or more of intra-mitochondrial sites, ii) hydrophobicity and positive charge, iii) ability to enter mitochondria via carrier proteins unique to the organelle, and iv) specific metabolism by mitochondrial enzymes. In another embodiment, by “mitochondria-penetrating moiety” or “mitochondria-targeting moiety” it is meant a molecule which utilizes “electrophoresis” of the vehicle and cargo into mitochondria at the expense of negative inside membrane potential. See, e.g., Belikova et al, FEBS Lett 2009 Jun. 18; 583(12): 1945-1950 and United States Patent Publication No. 2009/0099080,

The phosphatidylinositol-3-kinase (PI3K) pathway is well known to regulate a wide variety of essential cellular functions, including glucose metabolism, translational regulation of protein synthesis, cell proliferation, apoptosis, and survival. PI3K and the PI3K pathway are important players in tumor onset and maintenance. There are 3 classes of PI3K (I, II and III) categorized based on structure and substrate specificity. The class I PI3Ks are most tightly associated with human disease. Four class I PI3K isoforms have been identified: alpha (α), beta (β), delta (δ) and gamma (γ). The α and β PI3Ks are broadly expressed in human tissues and deregulation of these two PI3K family members occurs in many solid tumors. The δ and γ PI3Ks are primarily expressed in cells that comprise the human immune system and the δ PI3K is important for lymphomas and leukemia tumor cell growth. Thus, various PI3K inhibitors have been developed in recent years are available.

As used herein, “PI3K inhibitor” includes the specific PI3K inhibitor compounds described herein, and salts derived from pharmaceutically or physiologically acceptable acids, bases, alkali metals and alkaline earth metals. Physiologically acceptable acids include those derived from inorganic and organic acids. A number of inorganic acids are known in the art and include, without limitation, hydrochloric, hydrobromic, hydroiodic, sulfuric, nitric, and phosphoric acid. A number of organic acids are also known in the art and include, without limitation, lactic, formic, acetic, fumaric, citric, propionic, oxalic, succinic, glycolic, glucuronic, maleic, furoic, glutamic, benzoic, anthranilic, salicylic, tartaric, malonic, phenylacetic, mandelic, embonic, methanesulfonic, ethanesulfonic, panthenoic, benzenesulfonic, toluenesulfonic, stearic, sulfanilic, alginic, and galacturonic acids.

Some compounds, i.e., the Gamitrinib and/or PI3K inhibitor, may possess one or more chiral centers. Accordingly, the chemical compounds include each enantiomer, combinations of all possible enantiomers, diasteromers, racemers, and mixtures thereof. Where multiple chiral centers exist in the compounds described herein, also contemplated are each possible combinations of chiral centers within a compound, as well as all possible enantiomeric mixtures thereof. Those skilled in the art can prepare such optically active forms and resolve/synthesize racemic forms from their corresponding optically active forms.

Physiologically acceptable bases include those derived from inorganic and organic bases. A number of inorganic bases are known in the art and include, without limitation, aluminum, calcium, lithium, magnesium, potassium, sodium, and zinc sulfate or phosphate compounds, among others. Organic bases include, without limitation, N,N-dibenzylethylenediamine, chloroprocaine, choline, diethanolamine, ethylenediamine, meglumine, and procaine, among others.

Physiologically acceptable alkali salts and alkaline earth metal salts include, without limitation, sodium, potassium, calcium and magnesium salts, optionally in the form of esters, and carbamates.

The PI3K inhibitor compound salts can be also in the form of esters, carbamates, sulfates, ethers, oximes, carbonates, and other conventional “pro-drug” forms, which, when administered in such form, convert to the active moiety in vivo. In one embodiment, the prodrugs are esters.

The PI3K inhibitor compounds discussed herein also encompasses “metabolites” which are unique products formed by processing the PI3K inhibitor compound by the cell or subject. In one embodiment, metabolites are formed in vivo.

In one embodiment, the PI3K inhibitor is PX-866. The chemical name of PX-866 is acetic acid (1S,4E,10R,11R,13S,14R)-[4-diallylaminomethylene-6-hydroxy-1-methoxymethyl-10,13-dimethyl-3,7,17-trioxo-1,3,4,7,10,11,12,13,14,15,16,17-dodecahydro-2-oxa-cyclopenta[a]phenanthren-11-yl ester. PX-866 is a small-molecule wortmannin analogue inhibitor of the alpha, gamma, and delta isoforms of PI3K. PX-866 inhibits the production of the secondary messenger phosphatidylinositol-3,4,5-trisphosphate (PIP3) and activation of the PI3K/Akt signaling pathway, which may result in inhibition of tumor cell growth and survival in susceptible tumor cell populations. The molecular weight is 525.59 g/mol. PX-866 is available from Oncothyreon.

In one embodiment, the PI3K inhibitor is AZD6482. AZD6482 is a potent, selective and ATP competitive PI3Kβ inhibitor (see Nylander et al, Human target validation of phosphoinositide 3-kinase (PI3K)β: effects on platelets and insulin sensitivity, using AZD6482 a novel PI3Kβ inhibitor. J Thromb Haemost. 2012 October; 10(10):21.27-36, which is incorporated herein by reference). AZD6482 is a PI3Kβ inhibitor with IC50 of 10 nM, 8-,87- and 109-fold more selective to PI3Kβ than PI3Kδ, PI3Kα and PI3Kγ. The molecular weight is 408.45 g/mol. AZD6482 is an AstraZeneca compound available from Selleckchem.com.

In another embodiment, the PI3K inhibitor is LY294002. LY294002 is a morpholine-containing chemical compound that is a potent reversible inhibitor of PI3K. When used at a concentration of 50 μM it specifically abolished PI3 kinase activity (IC50=0.43 μg/ml, 1.40 μM) but did not inhibit other lipid and protein kinases such as PI4 kinase, PKC, MAP kinase or c-Src (Vlahos et al, A specific inhibitor of phosphatidylinositol 3-kinase, 2-(4-morpholinyl)-8-phenyl-4H-1-benzopyran-4-one (LY294002), J Biol Chem. 1994 Feb. 18;269(71:5241-8, which is incorporated herein by reference). The molecular weight is 307.34 g/mol. LY294002 is a Sigma compound available from Selleckchem.com.

In another embodiment, the PI3K inhibitor is BEZ235. BEZ235, also known as NVP-BEZ235, is an orally bioavailable imidazoquinoline targeting PI3K and the mammalian target of rapamycin (mTOR). BE235 inhibits PI3K kinase and mTOR kinase in the PI3K/AKT/mTOR kinase signaling pathway, which may result in tumor cell apoptosis and growth inhibition in PI3K/mTOR-overexpressing tumor cells. The molecular weight is 469.5 g/mol. BEZ235 is a Novartis compound available from Selleckchem.com.

In another embodiment, the PI3K inhibitor is GSK458. GSK458 is also known as GSK2126548. GSK458 is an oral, potent inhibitor of PI3K (α, β, γ, δ), mTORC1, and mTORC2. GSK458 has demonstrated broad anti-tumor activity against solid tumors and hematologic malignancies in vitro and in vivo. Cell lines with common activating mutations of PIK3CA are particularly sensitive to GSK458. The molecular weight is 505.5 g/mol. GSK458 is a GlaxoSmitliKline compound available from Selleckchem.com.

In another embodiment, the PI3K inhibitor is GDC0941. GDC0491 (also known as GDC-0491 and Pictilisib) is a potent, selective, orally bioavailable inhibitor of class I PI3 kinase. GDC0941 is designed to bind the ATP-binding pocket of PI3K and prevent formation of the second messenger phosphatidylinositol-3,4,5-triphosphate (PIP3), a key signaling intermediate that transmits signals downstream of PI3K. GDC-0941 is a potent inhibitor of PI3Kα/δ with IC50 of 3 nM, with modest selectivity against p110β (11-fold) and p110γ (25-fold). The molecular weight is 513.64 g/mol. GDC0941 is a Genentech/Roche compound available from Selleckchem.com. In one embodiment, the GDC0941 is the bismesylate salt.

In yet another embodiment, the PI3K inhibitor is ZSTK474. ZSTK474 is a potent, orally available inhibitor of class I PI3K isoforms with IC50 of 37 nM, mostly PI3Kδ. ZSTK474 at 1 μM potently reduces PI3K, activity to 4.7% of the control level (Yaguchi, JNCI, 2006, 98(8):545, which is incorporated herein by reference). Molecular modeling of the PI3K-ZSTK474 complex indicates that ZSTK474 could bind to the ATP-binding pocket of PI3K. The molecular weight is 417.41 g/mol. ZSTK474 is available from Selleckchem.com.

In another embodiment, the PI3K inhibitor is BKM120. BKM120, also known as NVM-BKM120 and Buparlisib, is an orally bioavailahle specific oral inhibitor of the pan-class I PI3K family of lipid kinases. BKM120 specifically inhibits class I PIK3 in the PI3K/AKT kinase (or protein kinase B) signaling pathway in an ATP-competitive manner, thereby inhibiting the production of the secondary messenger phosphatidylinositol-3,4,5-trisphosphate and activation of the PI3K signaling pathway. See, Maira et al, Identification and Characterization of NVP-BKM120, an Orally Available Pan-Class I PI3-Kinase Inhibitor, Identification and Characterization of NVP-BKM120, an Orally Available Pan-Class I PI3-Kinase Inhibitor, which is incorporated herein by reference. The molecular weight is 410.39 g/mol. BKM120 is a Novartis compound available from Selleckchem.com.

In yet another embodiment, the PI3K inhibitor is GSK2636771 (or GSK236771B). GSK2636771 is a potent, orally bioavailable, PI3Kβ-selective substituted benzimidazole inhibitor, sensitive to PTEN null cell lines. The molecular weight is 433.42. GSK2636771 is a GlaxoSmithKline compound available from Selleckchem.com.

In yet another embodiment, the PI3K inhibitor is GDC-0980, also called Apitolisib. GDC-0980 is a novel class I PI3K/mTOR kinase inhibitor with robust activity in cancer models driven by the PI3K pathway. See, Wallin et al, Mol Cancer Ther. 2011 December;10(12):2426-36, which is incorporated herein by reference. The molecular weight is 498.6. Apitolisib is available from Selleckchem.com.

In another embodiment, the PI3K inhibitor is GSK2126458 (or GSK458 or Omipalisib). Omipalisib (GSK2126458, GSK458) is a highly selective and potent inhibitor of p110α/β/δ/γ, mTORC1/2 with Ki of 0.019 nM/0.13 nM/0.024 nM/0.06 nM and 0.18 nM/0.3 nM, respectively. See, Knight et al, Discovery of GSK2126458, a Highly Potent Inhibitor of PI3K and the Mammalian Target of Rapamycin, ACS Med. Chem. Lett., 2010, 1 (1), pp 39-43, which is incorporated by reference herein. The molecular weight is 505.5. GSK2126458 is a GlaxoSmithKline compound available from Selleckchem.com.

A pharmaceutical composition comprising gamitrinib and a PI3K inhibitor described herein is generally formulated to be compatible with its intended route of administration. In one embodiment, the composition includes a pharmaceutically acceptable carrier or diluent. Examples of routes of administration include parenteral, e.g., intravenous, intradermal, subcutaneous, inhalation (oral, tranasal, and intratracheal), ocular, transdermal (topical), subligual, intracrainial, epidural, vaginal, intraperitoneal, intratumoral, intranodal, transmucosal, and rectal administration. Routes of administration may be combined, if desired. In some embodiments, the administration is repeated periodically.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as EDTA; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injectable use include sterile aqueous solutions (where water soluble) or dispersions, and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required panicle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be achieved by including an agent which delays absorption, e.g., aluminum monostearate or gelatin, in the composition.

Sterile injectable solutions can be prepared by incorporating an active compound (e.g., Gamitrinib and/or a PI3K inhibitor) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Although the composition may be administered alone, it may also be administered in the presence of one or more pharmaceutical carriers that are physiologically compatible. The carriers may be in dry or liquid form and must be pharmaceutically acceptable. Liquid pharmaceutical compositions are typically sterile solutions or suspensions. When liquid carriers are utilized for parenteral administration, they are desirably sterile liquids. Liquid carriers are typically utilized in preparing solutions, suspensions, emulsions, syrups and elixirs. In one embodiment, the composition may be combined with a liquid carrier. In another embodiment, the composition may be suspended in a liquid carrier. One of skill in the art of formulations would be able to select a suitable liquid carrier, depending on the route of administration. The composition may alternatively be formulated in a solid carrier. In one embodiment, the composition may be compacted into a unit dose form, i.e., tablet or caplet. In another embodiment, the composition may be added to unit dose form, i.e., a capsule. In a further embodiment, the composition may be formulated for administration as a powder. The solid carrier may perform a variety of functions, i.e., may perform the functions of two or more of the excipients described below. For example, the solid carrier may also act as a flavoring agent, lubricant, solubilizer, suspending agent, filler, glidant, compression aid, binder, disintegrant, or encapsulating material.

Oral compositions generally include an inert diluent or an edible carrier. For the purpose of oral therapeutic administration, the active compound can be incorporated with excipients and used in the form of tablets, troches, gel tab, dispersible powder, granule, suspension, liquid, thin film, chewable tablet, rapid dissolve tablet, medical lollipop, or fast melt or capsules, e.g., gelatin capsules. Oral compositions can also be prepared using a fluid carrier for use as a mouthwash. One of skill in the art would readily be able to formulate the compositions discussed herein in any one of these forms.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL™, or corn starch; a lubricant such as magnesium stearate; a glidant such as colloidal silicon dioxide; a sweetening agent such as sucrose or saccharin; syrup; coloring agent; coating; emulsifier; emollient; encapsulating material; granulating agent; metal chelator; osmo-regulator, pH adjustor; preservative; solubilizer; sorbent; stabilizer; surfactant; suspending agent; thickener; viscosity regulator; or a flavoring agent such as peppermint, methyl salicylate, or orange flavoring. See, for example, the excipients described in the “Handbook of Pharmaceutical Excipients”, 5^{th} Edition, Eds.: Rowe, Sheskey, and Owen, APhA Publications (Washington, D.C.), Dec. 14, 2005, which is incorporated herein by reference.

For administration by inhalation, a compound is delivered in the form of an aerosol spray from pressured container or dispenser that contains a suitable propellant, e.g., a gas or liquified propellant, e.g., dichlorodifluoromethane, such as carbon dioxide, nitrogen, propane and the like or a nebulizer. Also provided is the delivery of a metered dose in one or more actuations.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In another embodiment, the composition may be utilized as an inhalant. For this route of administration, the composition may be prepared as fluid unit doses containing Gaminitrab and PI3K inhibitor and a vehicle for delivery by an atomizing spray pump or by dry powder for insufflation.

In a further embodiment, the composition may be administered by a sustained delivery device. “Sustained delivery” as used herein refers to delivery of the composition which is delayed or otherwise controlled. Those of skill in the art are aware of suitable sustained delivery devices. For use in such sustained delivery devices, the composition is formulated as described herein. In one embodiment, the compounds may be formulated with injectable microspheres, bio-erodible particles, polymeric compounds (polylactic or polyglycolic acid), beads, liposomes, or implantable drug delivery devices.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

As discussed above, compositions useful herein contain Gamitrinib and a PI3K inhibitor in a pharmaceutically acceptable carrier optionally with other pharmaceutically inert or inactive ingredients. In another embodiment, Gamitrinib and a PI3K inhibitor are present in a single composition. In a further embodiment, Gamitrinib and a PI3K inhibitor are combined with one or more excipients and/or other therapeutic agents as described below.

The composition may be administered on regular schedule, i.e., daily, weekly, monthly, or yearly basis or on an irregular schedule with varying administration days, weeks, months, etc. Alternatively, administration of the composition may vary. In one embodiment, the first dose of the composition is higher than the subsequent doses. In another embodiment, the first dose containing the composition is lower than subsequent doses. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The composition may be formulated neat or with one or more pharmaceutical carriers for administration. The amount of the pharmaceutical carrier(s) is determined by the solubility and chemical nature of the components of the composition, chosen route of administration and standard pharmacological practice. The pharmaceutical carrier(s) may be solid or liquid and may include both solid and liquid carriers. A variety of suitable liquid carriers is known and may be selected by one of skill in the art. Such carriers may include, e.g., DMSO, saline, buffered saline, hydroxypropylcyclodextrin, and mixtures thereof. Similarly, a variety of solid carriers and excipients are known to those of skill in the art.

As used herein, the term “effective amount” or “pharmaceutically effective amount” as it refers to individual composition components, refers to the amount of Gamitrinib or the selected PI3K inhibitor described herein that elicits the biological or medicinal response in a tissue, system, animal, individual or human that is being sought by a researcher, veterinarian, medical doctor or other clinician, which includes one or more of the following, preventing a disease; e.g., inhibiting a disease, condition or disorder in an individual that is experiencing or displaying the pathology or symptomatology of the disease, condition or disorder (i.e., arresting or slowing further development of the pathology and/or symptomatology); ameliorating a disease, condition or disorder in an individual that is experiencing or displaying the pathology or symptomatology of the disease, condition or disorder (i.e., reversing the pathology and/or symptomatology); and inhibiting a physiological process. For example, an effective amount of a combination of Gamitrinib and a selected PI3K inhibitor, when administered to a subject to treat cancer, is sufficient to inhibit, slow, reduce, or eliminate tumor growth in a subject having cancer.

The effective dosage or amount of the compounds may vary depending on the particular compound employed, the mode of administration, the type and severity of the condition being treated, and subject being treated as determined by the subject's physician. The effective dosage of each active component (e.g., Gamitrinib and a PI3K inhibitor) is generally individually determined, although the dosages of each compound can be the same. In one embodiment, the dosage is about 0.1 μg to about 1000 mg. In one embodiment, the effective amount is about 0.1 to about 50 mg/kg of body weight including any intervening amount. In another embodiment, the effective amount is about 0.5 to about 40 mg/kg. In a further embodiment, the effective amount is about 0.7 to about 30 mg/kg. In still another embodiment, the effective amount is about 1 to about 20 mg/kg. In yet a further embodiment, the effective amount is about 0.001 mg/kg to 1000 mg/kg body weight. In another embodiment, the effective amount is less than about 5 g/kg, about 500 mg/kg, about 400 mg/kg, about 300 mg/kg, about 200 mg/kg, about 100 mg/kg, about 50 mg/kg, about 25 mg/kg, about 10 mg/kg, about 1 mg/kg, about 0.5 mg/kg, about 0.25 mg/kg, about 0.1 mg/kg, about 100 μg/kg, about 75 μg/kg, about 50 μg/kg, about 25 μg/kg, about 10 μg/kg, or about 1 μg/kg. However, the effective amount of the compound can be determined by the attending physician and depends on the condition treated, the compound administered, the route of delivery, age, weight, severity of the patient's symptoms and response pattern of the patient.

Toxicity and therapeutic efficacy of the compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue, e.g., bone or cartilage, in order to minimize potential damage to uninfected cells and, thereby, reduce side effects.

The data obtained from cell culture assays (such as those described in the examples below) and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

One or more of the compounds discussed herein may be administered in combination with other pharmaceutical agents, as well as in combination with each other. The term “pharmaceutical” agent as used herein refers to a chemical compound which results in a pharmacological effect in a patient. A “pharmaceutical” agent can include any biological agent, chemical agent, or applied technology which results in a pharmacological effect in the subject.

In addition to the components described above, the compositions may contain one or more medications or therapeutic agents which are used to treat solid tumors. In one embodiment, the medication is a chemotherapeutic. Examples of chemotherapeutics include those recited in the “Physician's Desk Reference”, 64^{th} Edition, Thomson Reuters, 2010, which is hereby incorporated by reference. Therapeutically effective amounts of the additional medication(s) or therapeutic agents are well known to those skilled in the art. However, it is well within the attending physician to determine the amount of other medication to be delivered.

In one embodiment, the chemotherapeutic is selected from among cisplatin, carboplatin, 5-fluorouracil, cyclophosphamide, oncovin, vincristine, prednisone, or rituximab, mechlorethamine, cyclophosphamide, ifosfamide, melphalan, chlorambucil, carmustine, lomustine, semustine, thriethylenemelamine, triethylene thiophosphoramide, hexamethylmelamine altretamine, busulfan, triazines dacarbazine, methotrexate, trimetrexate, fluorodeoxyuridine, gemcitabine, cytosine arabinoside, 5-azacytidine, 2,2′-difluorodeoxycytidine, 6-mercaptopurine, 6-thioguanine, azathioprine, 2′-deoxycoformycin, erythrohydroxynonyladenine, fludarabine phosphate, 2-chlorodeoxyadenosine, camptothecin, topotecan, irinotecan, paclitaxel, vinblastine, vincristine, vinorelbine, docetaxel, estramustine, estramustine phosphate, etoposide, teniposide, mitoxantrone, mitotane, or aminoglutethimide.

In one embodiment, the compound is combined with one or more of these pharmaceutical agents, i.e delivered to the patient concurrently. In another embodiment, the compound is administered to the patient concurrently therewith one or more of these pharmaceutical agents. In a further embodiment, the compound is administered prior to one or more of these pharmaceutical agents. In still another embodiment, the compound is administered subsequent to one or more of these pharmaceutical agents.

These pharmaceutical agents may be selected by one of skilled in the art and thereby utilized in combination with Gaminitrib and/or a PI3K inhibitor. Examples of these additional agents include, without limitation, cytokines (interferon (α, β, γ) and interleukin-2), lymphokines, growth factors, antibiotics, hacteriostatics, enzymes (L-asparaginase), biological response modifiers (interferon-alpha; IL-2; G-CSF; and GM-CSF), differentiation agents (retinoic acid derivatives), radiosensitizers (metronidazole, misonidazole, desmethylmisonidazole, pimonidazole, etanidazole, nimorazole, RSU 1069, E09, RB 6145, SR4233, nicotinamide, 5-bromodeozyuridine, 5-iododeoxyuridine, bromodeoxycytidine), hormones (adrenocorticosteroids, prednisone, dexamethasone, aminoglutethimide), progestins (hydroxyprogesterone caproate, medroxyprogesterone acetate, megestrol acetate), estrogens (diethylstilbestrol, ethynyl estradiol/equivalents), antiestrogens (tamoxifen), androgens (testosterone propionate, fluoxymesterone), antiandrogens (flutamide, gonadotropin-releasing hormone analogs, leuprolide), photosensitizers (hematoporphyrin derivatives, Photofrin®, benzoporphyrin derivatives, Npe6, tin etioporphyrin, pheoboride-α, bacteriochlorophyll-α, naphthalocyanines, phthalocyanines, and zinc phthalocyanines), proteosome inhibitors (bortezomib), tyrosine kinase inhibitors (imatinib mesylate, dasatinib, nilotinib, MK-0457, and Omacetaxine), immunotherapeutics, vaccines, biologically active agents, or HSP90 inhibitors.

### III. Kits

Also provided herein are kits or packages of compositions containing Gamitrinib and a PI3K inhibitor. The kits may be organized to indicate a single formulation or combination of formulations to be taken at each desired time.

Suitably, the kit contains packaging or a container with Gamitrinib and a PI3K inhibitor formulated for the desired delivery route. In one embodiment, the kit contains instructions on dosing and an insert regarding the active agent(s). In another embodiment, the kit may further contain instructions for monitoring circulating levels of the components of the composition and materials for performing such assays including, e.g., reagents, well plates, containers, markers or labels, and the like. Such kits are readily packaged in a manner suitable for treatment of a desired indication. Other components for inclusion in the kits will be readily apparent to one of skill in the art, taking into consideration the desired indication and the delivery route.

The compositions described herein can be a single dose or for continuous or periodic discontinuous administration. For continuous administration, a package or kit can include Gamitrinib and a PI3K inhibitor in each dosage unit (e.g., solution, lotion, tablet, pill, or other unit described above or utilized in drug delivery), and optionally instructions for administering the doses daily, weekly, or monthly, for a predetermined length of time or as prescribed. When the composition is to be delivered periodically in a discontinuous fashion, a package or kit can include placebos during periods when the composition is not delivered. When varying concentrations of the composition, the components of the composition, or the relative ratios of Gamitrinib and/or a PI3K inhibitor within the composition over time is desired, a package or kit may contain a sequence of dosage units which provide the desired variability.

A number of packages or kits are known in the art for dispensing the compositions for periodic oral use. In one embodiment, the package has indicators for each period. In another embodiment, the package is a labeled blister package, dial dispenser package, or bottle. The composition may also be sub-divided to contain appropriate quantities of Gaminitrab and PI3K inhibitor. For example, the unit dosage may be packaged compositions, e.g., packeted powders, vials, ampoules, prefilled syringes or sachets containing liquids.

The packaging means of a kit may itself be geared for administration, such as an inhalant, syringe, pipette, eye dropper, or other such apparatus, from which the formulation may be applied to an affected area of the body, such as the lungs, injected into a subject, or even applied to and mixed with the other components of the kit.

The compositions also may be provided in dried or lyophilized forms. When reagents or components are provided as a dried form, reconstitution generally is by the addition of a suitable solvent. Such formulations can be stored either in a ready-to-use form or in a form requiring reconstitution prior to administration. The formulations may also be contained with a container having a sterile access port, for example, an intravenous solution bag or vial having a stopper pierceable by a hypodermic injection needle. It is envisioned that the solvent also may be provided in another package.

The kits also may include a means for containing the vials in close confinement for commercial sale such as, e.g., injection or blow-molded plastic containers into which the desired vials are retained. The kits may further include, or be packaged with a separate instrument for assisting with the injection/administration or placement of the composition within the body of an animal. Such an instrument may be an inhaler, syringe, pipette, forceps, measuring spoon, eye dropper or any such medically approved delivery means.

In one embodiment, a kit is provided and contains Gamitrinib and a PI3K inhibitor. These components may be in the presence or absence of one or more of the carriers or excipients described above. The kit may optionally contain instructions for administering the composition to a subject.

In a further embodiment, a kit is provided and contains Gamitrinib in a first dosage unit, a PI3K inhibitor in a second dosage unit, and one or more of the carriers or excipients described above in a third dosage unit. The kit may optionally contain instructions for administration.

### IV. Methods

One aspect of the invention provides a method of treating cancer in a subject in need thereof. This aspect is based on the inventor's discovery that the combination of Gamitrinib with certain PI3K inhibitors reversed tumor reprogramming induced by administering PI3K alone, and potently enhances anticancer activity.

In one embodiment, the method of treating cancer in a subject in need thereof includes administering a pharmaceutical composition comprising Gamitrinib and a selected PI3K inhibitor. The pharmaceutical composition may be any composition as described herein. In one embodiment, the PI3K inhibitor is PX-866. In another embodiment, the PI3K inhibitor is AZD6482. In another embodiment, the PI3K inhibitor is LY294002. In another embodiment, the PI3K inhibitor is BEZ235. In another embodiment, the PI3K inhibitor is GSK458. In another embodiment, the PI3K inhibitor is GDC0941. In another embodiment, the PI3K inhibitor is ZSTK474. In another embodiment, the PI3K inhibitor is BKM120. In another embodiment, the PI3K inhibitor is GSK2636771. In another embodiment, the PI3K inhibitor is GSK458. In another embodiment, the PI3K, inhibitor is GDC-0980.

In one embodiment, the cancer being treated is any of those described herein or which may be benefitted by the treatment of a PI3K inhibitor and gamitrinib co-therapy. In one embodiment, the method includes the treatment of cancer and tumor cells selected from, but not limited to, breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, acute and chronic lymphocytic and myelocytic leukemia, myeloma, Hodgkin's and non-Hodgkin's lymphoma, and multidrug resistant cancer. In one embodiment, the cancer is a drug resistant cancer.

The therapeutic compositions administered in the performance of these methods, e.g., Gamitrinib and a selected PI3K inhibitor, may be administered directly into the environment of the targeted cell undergoing unwanted proliferation, e.g., a cancer cell or targeted cell (tumor) microenvironment of the patient. In an alternative embodiment, the compositions are administered systemically, without regard to the location of the cancer, i.e., parenteral administration. Conventional and pharmaceutically acceptable routes of administration include, but are not limited to, systemic routes, such as intraperitoneal, intravenous, intranasal, intravenous, intramuscular, intratracheal, subcutaneous, and other parenteral routes of administration or intratumoral or intranodal administration, as discussed above. Routes of administration may be combined, if desired. In some embodiments, the administration is repeated periodically. Dosages may be administered continuously for a certain period of time, or periodically every week, month, or quarter, dependent on the condition and response of the patient, as determined by a physician.

In one embodiment, the compositions i.e., Gamitrinib and a selected PI3K inhibitor, are administered at the same time. In another embodiment, the compositions are administered sequentially. In another embodiment, Gamitrinib is administered first. In another embodiment, the PI3K inhibitor is administered first. In another embodiment, the compositions are administered within a suitable period, e.g., hours, days or weeks of each other. These periods may be determined by a physician. In one embodiment, the compositions are administered periodically, e.g. every day, week, two weeks, monthly, quarterly, or as prescribed by physician.

These therapeutic compositions may be administered to a patient preferably suspended in a biologically compatible solution or pharmaceutically acceptable delivery vehicle, as discussed herein. The various components of the compositions are prepared for administration by being suspended or dissolved in a pharmaceutically or physiologically acceptable carrier such as isotonic saline; isotonic salts solution or other formulations that will be apparent to those skilled in such administration. The appropriate carrier will be evident to those skilled in the art and will depend in large part upon the route of administration. Other aqueous and non-aqueous isotonic sterile injection solutions and aqueous and non-aqueous sterile suspensions known to be pharmaceutically acceptable carriers and well known to those of skill in the art may be employed for this purpose.

In one embodiment, the methods described herein include administration of Gamitrinib and a PI3K inhibitor, as described above, in combination with other known anti-proliferative disease therapies. In one embodiment of such combination therapy, the present method can include administration of a passive therapeutic that can immediately start eliminating the targeted cell undergoing unrestricted or abnormal replication or proliferation, e.g., tumor. This can also be accompanied by administration of active immunotherapy to induce an active endogenous response to continue the tumor eradication. Such immune-based therapies can eradicate residual disease and activate endogenous antitumor responses that persist in the memory compartment to prevent metastatic lesions and to control recurrences. This treatment may occur, before, during or after administration of Gamitrinib and/or PI3K inhibitor. In another example, surgical debulking, in certain embodiments is a necessary procedure for the removal of large benign or malignant masses, and can be employed before, during or after application of the methods and compositions as described herein. Chemotherapy and radiation therapy, in other embodiments, bolster the effects of the methods described herein. Such combination approaches (surgery plus chemotherapy/radiation plus immunotherapy) with the methods of administering Gamitrinib and a PI3K inhibitor are anticipated to be successful in the treatment of many proliferative diseases.

Still other adjunctive therapies for use with the methods and compositions described herein include non-chemical therapies. In one embodiment, the adjunctive therapy includes, without limitation, acupuncture, surgery, chiropractic care, passive or active immunotherapy, X-ray therapy, ultrasound, diagnostic measurements, e.g., blood testing. In one embodiment, these therapies are be utilized to treat the patient. In another embodiment, these therapies are utilized to determine or monitor the progress of the disease, the course or status of the disease, relapse or any need for booster administrations of the compounds discussed herein.

### IV. EXAMPLES

The invention is now described with reference to the following examples. These examples are provided for the purpose of illustration only. The compositions, experimental protocols and methods disclosed and/or claimed herein can be made and executed without undue experimentation in light of the present disclosure. The protocols and methods described in the examples are not considered to be limitations on the scope of the claimed invention. Rather this specification should be construed to encompass any and all variations that become evident as a result of the teaching provided herein. One of skill in the art will understand that changes or variations can be made in the disclosed embodiments of the examples, and expected similar results can be obtained. For example, the substitution of reagents that are chemically or physiologically related for the reagents described herein are anticipated to produce the same or similar results. All such similar substitutes and modifications are apparent to those skilled in the art and fall within the scope of the invention.

### Example 1: Materials and Methods

Informed consent was obtained from all patients and the study was approved by an Institutional Review Board. The clinico-pathologic characteristics of patient samples used for organotypic culture¹⁰ are presented in Table 1. All animal experiments were approved by an Institutional Animal Care and Use Committee. Methods for cell culture, analysis of bioenergetics, kinase assays, CypD function, tumor cell invasion and metabolomics, reverse-phase protein array and high-throughput screening are presented in the Extended Methods section. Data were analyzed using two-sided unpaired t tests. Data are mean±SEM. A p value of ≤0.05 was considered statistically significant.

Patients. Fresh, patient-derived tissues obtained from surgical resections of colon adenocarcinoma (1 case), infiltrating ductal breast adenocarcinoma (4 cases), non-small cell lung adenocarcinoma (4 cases), and grade IV glioblastoma (5 cases) were used in this study. Informed consent was obtained from all patients and the study was approved by the Institution Review Board at the Fondazione IRCCS Ca′ (Ganda hospital (Milan, Italy). The clinicopathological characteristics of the patient series used in this study are presented in Table 1 below.

TABLE 1

| | | | | | | | | Clinico- |
|---|---|---|---|---|---|---|---|---|
| Ex vivo | | HISTO | | | DIAG- | | | pathological |
| treatment | Organ | N. | SEX | AGE | NOSIS | GRADE | TMN | characteristics |
| LY294002 | COLON | 08- | M | 62 | AdCa | G2 | pT3N1 | |
| | | 21047 | | | | | | |
| | LUNG | 13-I- | M | 73 | NSCLC | G2 | pT1bN0 | |
| | | 09438 | | | (AdCa) | | | |
| | BREAST | 13-I- | F | 50 | IDC | G3 | pT3pN3a | ER+, PgR+, |
| | | 09617 | | | | | | C-ErbB2− |
| | BRAIN | 13-I- | F | 70 | Anaplastic | III | | MGMT (M), |
| | | 09668 | | | Astr. | | | 1p/19q LOH |
| | | | | | | | | neg |
| | BREAST | 13-I- | F | 63 | IDC | G2 | pt2aN1a | ER−, PgR−, |
| | | 09828 | | | | | | C-ErbB2− |
| PX-866- | BRAIN | 13-I- | F | 31 | GBM | IV | | MGMT (M), |
| Gamitrinib | | 11647 | | | | | | 1p/19q LOH− |
| | BRAIN | 13-I- | F | 73 | GBM | IV | | MGMT (UM), |
| | | 11712 | | | | | | 1p/19g LOH− |
| | BREAST | 13-I- | F | 43 | IDC | G3 | pT2aN0 | ER+, PgR+, |
| | | 11754 | | | | | | C-ErbB2− |
| | LUNG | 13-I- | M | 81 | NSCLC | G2 | pT2aN1 | |
| | | 11984 | | | (AdCa) | | | |
| | BREAST | 13-I- | F | 54 | IDC | G2 | pT2N0 | ER+, PgR+, |
| | | 11994 | | | | | | C-ErbB2− |
| | LUNG | 13-I- | M | 65 | NSCLC | G2 | pTIbN0 | |
| | | 12382 | | | (AdCa) | | | |
| | BRAIN | 13-I- | M | 66 | GBM | IV | | MGMT (M), |
| | | 13080 | | | | | | Ip/19q LOH- |
| | BRAIN | 13-I | F | 64 | GBM | IV | | MGMT |
| | | 13356 | | | | | | (M), 1p/19q |
| | | | | | | | | LOH+ |

NSCLC, non small cell lung cancer;
GBM, glioblastoma;
IDC, infiltrating ductal carcinoma;
Astr, astrocytoma;
AdCa, adenocarcinoma;
neg, negative.

Organotypic cultures. Short-term organotypic cultures from the different patient samples were established as described previously. Briefly, precise thick tissue slices (300 μm) were obtained from each case by vibratome serial cutting (VT1200, Leica Microsystems, Milan, Italy), and cultured in six-well plates on organotypic inserts (Milken PICM ORG, Merck Millipore) for up to 48 h in 1 nil of Ham-F12 complete medium supplemented with vehicle (DMSO, 2.5 μl), pan-PI3K inhibitor LY294002 (50 or 100 μM) or PX-886 (2.5, 5 or 10 μM), or mitochondrial-targeted small molecule Heat Shock Protein-90 (Hsp90) inhibitor, Gamitrinib (GA mitochondrial matrix inhibitor, 10 or 25 μM), or the combination of PX-886 plus Gamitrinib (each agent used at 10 μM). At the end of the experiment, one tissue slice per condition was formalin-fixed and paraffin-embedded (FFPE), and processed for morphological and immunohistochemical analysis. An additional tissue slice collected at the end of the experiment was embedded in optimal cutting temperature, and snap-frozen for molecular or immunofluorescence studies.

Cell culture. Human glioblastoma LN229, U87 and U251, prostate adenocarcinoma PC3, cervical carcinoma HeLa, and breast adenocarcinoma MCF-7 cells were obtained from the American Type Culture Collection (ATCC, Manassas, Va.), and maintained in culture according to the supplier's specifications. Human glioblastoma U87 cells stably transfected with luciferase (U87-Luc) have been described in previous studies. WT or CypD^{-/-} MEFs were characterized earlier. LN229 cells with stable short hairpin RNA (shRNA) knockdown of CypD have been characterized in previous studies.

Antibodies and reagents. The antibodies to hexokinase-I (HK-I, Cell Signaling), hexokinase-II (HK-II, Cell Signaling), Cox-IV (Cell Signaling), LC-3 (Cell Signaling), Ser473-phosphorylated Akt (Cell Signaling), Thr308-phosphotylated Akt (Cell Signaling), Akt (Cell Signaling), Tyr397-phosphorylated FAK (Invitrogen), Tyr925-phosphorylated FAK (Cell Signaling), FAK (Cell Signaling), VDAC (Cell Signaling), CypD (Calbiochem), β-tubulin (Sigma-Aldrich), and β-actin (Sigma-Aldrich) were used. Small interfering RNA (siRNA) directed to PI3K p110α subunit was from Santa Cruz Biotechnology. The complete chemical synthesis, HPLC profile, and mass spectrometry of mitochondrial-targeted Hsp90 antagonist, Gamitrinib has been reported. The Gamitrinib variant containing triphenylphosphonium (Gamitrinib-TPP-OH) as a mitochondrial-targeting moiety was used throughout this study, except for in vivo combination studies with the dual PI3K inhibitor, NVP-BEZ-235 (BEZ), where the Gamitrinib variant containing four tandem repeats of guanidinium (Gamitrinib-G4) as a mitochondrial-targeting moiety was used. Non-mitochondrially directed Hsp90 inhibitor, 17-AAG was obtained from LC-Laboratories.

Subcellular and submitochondrial fractionation. Mitochondrial fractions were isolated using an ApoAlert™ cell fractionation kit (CLONTECH), as described. Subinitochondrial fractions comprising outer membrane (OM), inner membrane (IM), inter-membrane space (IMS) and matrix were prepared as described.

Mitochondrial integrity and cell viability. LN229 or U251 cells were treated with vehicle (DMSO), PI3K inhibitors, BEZ (0.5 μM) or LY294002 (50 μM), alone or combination with Gamitrinib (0.5-5 μM, respectively) for 24 h, and analyzed for changes in mitochondrial membrane potential by JC-1 staining and multiparametric flow cytometry, as described. For cell viability, LN229, U87 or U251 cells treated with vehicle, PI3K inhibitors BEZ or LY294002, or, alternatively, transfected with control cDNA or Δ-p85 dominant negative PI3K mutant, alone or in combination with Gamitrinib, were examined by a 3 (4,5-dimethyl-thyazoyl-2-yl)2,5 diphenyltetrazolium bromide (MTT) colorimetric assay. Changes in cell viability in LN229 cells exposed to PX-866 (0-25 μM) were monitored by Trepan blue staining, or, alternatively, MTT. For apoptosis, LN229 cells)(1×10⁶) treated with vehicle, Gamitrinib or PI3K inhibitors (BEZ, LY294002), alone or in combination for 24 h, were labeled for annexin V plus propidium iodide (PI) (BD Biosciences), and analyzed by multiparametric flow cytometry (BD), as described. Alternatively, cytosolic fractions isolated from treated tumor cells were analyzed for released cytochrome c, by Western blotting. For analysis of colony-forming ability, PC3 or LN229 cells were treated with vehicle (Veh) or PX-866 (10 μM) and analyzed in a colony formation assay after 12 days. Colonies were stained with crystal violet. In some experiments, LN229 cells treated with PX-866 (0-25 μM) were analyzed for DNA content by PI staining and flow cytometry.

Analysis of bioenergetics and PPlase activity. LN229 or PC3 cells treated with vehicle (DMSO) or PI3K inhibitors PX-866 (10 μM), AZD6482 (10 μM) or GDC0941 (2 μM) for 48 h were analyzed for ATP production, glucose utilization, lactate generation, HK activity and oxygen consumption, as described². CypD PPIase activity was determined colorimetrically with chymotrypsin using the synthetic tetrapeptide N-Succinyl-Ala-Ala-Pro-Phe p-nitroanilide (Sigma) in which the rate of conversion of cis to trans of a proline residue makes it susceptible to cleavage by chymotrypsin, resulting in the release of the chromogenic dye, p-nitroanilide. Eight-hundred-fifty of 0.1 M Tris-Hcl (pH 8.0) and 40 μl of 200 μM solution of α-chymotrypsin were mixed in the spectrometer cell and preincubated at 0° C. for 10 min. Forty μg of WT or mutant CypD protein solution extracted after plasmid transfection in CypD^{-/-} MEFs was added, and after 5 min, the assay was initiated by adding 10 μl of a 7.8 mM solution of the peptide dissolved in a 0.47 M LiCl in trifluoroethanol. Cis-to-trans isomerization of the Ala-Pro peptide bond, coupled with the chymotryptic cleavage of the trans peptide, was quantified by increase in absorbance at 380 nm for a 40 s in a spectrophotometer. The temperature was maintained to 0° C. throughout the assay. HK-II activity in reconstituted LN229 cells was determined as described.

Plasmids and Mutagenesis. A PPlase-defective CypDHis168→, Gln mutant was characterized previously, and validated in recent studies. A PI3K Δ-p85 dominant negative mutant was as described. Full length (FLAG-tagged) or mature CypDcDNAs were described. Substitution of predicted CypD phosphorylation sites Ser31→Ala, Ser123→Ala, and Ser31/123→Ala in CypD was carried out using QuikChange Site-Directed Mutagenesis Kit (Stratagene) with oligonucleotides (mutated sequences underlined):

SEQ ID NO: 1
5′-GCG GCC CGC GCC TGC GCC AAG GGC TCC GGC GAC-3'
(Ser31, AGC → GCC),
SEQ ID NO: 2
5′-CGG GAA GTC CAT CTA CGG AGC CCG CTT TCC TGA CGA
GAA CT-3'(Ser123, AGC → GCC).

Mutant constructs were confirmed by DNA sequencing and transfected in CypD-depleted cells for reconstitution experiments. In some experiments, CypD depleted LN229 cells were transfected with WT or mutant CypD constructs and analyzed for bioenergetics (glucose utilization, oxygen consumption, ATP production) or cell viability (JC1 or Annexin V multiparametri staining, cytochrome c release) readouts. Alternatively, CypD-depleted LN229 cells were transfected with FLAG-tagged full length CypD, treated with vehicle (DMSO) or PX-866 (10 μM for 48 h), and immunoprecipitated with anti-FLAG-M2 affinity gel, followed by Western blotting.

Tumor cell invasion and 3D spheroids. Eight μm filters of Transwell migration chambers (Corning Life Sciences, Lowell, Mass.) were coated with 150 μl of 80 μg/ml reconstituted basement membrane or Matrigel (Becton Dickinson, Franklin Lakes, N.J.). PC3 cells were treated with PX-866 (10 μM) for 48 h, and seeded onto the coated Transwell filters at a density of 1.5×10⁵ cells/well in media containing 2% FCS. Media containing 20% FCS was placed in the lower chamber as chemoattractant. Cells were allowed to invade and adhere to the lower chamber, and stained using crystal violet. Cell invasion was quantified using Image Pro Plus 7 after additional 24-48 h, as described. For analysis of 3D spheroids, tissue culture treated 96-well plates were coated with 50 μl 1% DIFCO Agar Noble (Becton Dickinson). LN229 cells were seeded at 5,000 cells/well and allowed to form spheroids over 72 h. Spheroids were harvested, treated with PX-866 (0-10 μM) and placed in a collagen plug containing EMEM, FBS, L-Glutamine, sodium bicarbonate, and collagen type I (Gibco, 1.5 mg/ml). The collagen plug was allowed to set and 1 ml RPMI with 10% FBS or DMEM with 5% FBS was added to the top of the plug. Cell invasion was analyzed every 24 h and quantified using Image Pro Plus 7, as described.

Kinase assays. These experiments were carried out as basically described previously. GST or GST-conjugated proteins were incubated with active Akt (0.1 μg/μL) in 25 mmol/L MOPS, 12.5 mmol/L β-glycerolphosphate, 25 mmol/L MgCl₂, 5 mmol/L EGTA, 2 mmol/L EDTA, 0.25 mmol/L DTT, and 0.1 mmol/L ATP with or without ³²P-γATP for 20 min at 30° C., followed by autoradiography. GSK3β was used as a control Akt substrate.

High-throughput screening and synergy experiments. Compounds for the molecular targeted cancer therapeutics were either purchased from SelleckChem or acquired under Material Transfer Agreement (MTA) with the Wistar Institute MTA. Compounds were suspended in DMSO. The purity and identity of the compounds was confirmed by LC/MS for a fraction of the compounds. Source plates with a 10 point dilution series (5-fold dilutions, ranging from 10-0.0001 μmol/L) were prepared for screening. For these experiments, LN229 cells (17,000/cm²) were treated simultaneously with 1 μM Gamitrinib and the indicated compounds (concentration range 0.0005-10 μmol/L) for 18 h at 37° C. Cell viability was determined by the addition of resazurin to a final concentration of 50 μM and incubation for 8 h. Fluorescence intensity (excitation, 560 nm, emission, 590 nm) was determined on an Envision Xcite Multilabel Reader (Perkin-Elmer), and the fractional growth inhibition was determined by normalizing assay wells to the aggregated average responses of positive control (10 μM doxorubicin) and negative control (0.2% DMSO) treatments (n=12). The screen was repeated twice with comparable results.

A synergistic anticancer activity of the combination of Gamitrinib and selected kinase inhibitors was further assessed by Bliss independence analysis. For these experiments, LN229 cells were treated with Gamitrinib (concentration range 2-0.008 μmol/L) and selected kinase inhibitors (concentration range 2-0.008 μmol/L) in a 7×7 matrix for 18 h at 37° C. Cell viability was measured by addition of resazurin, and the fractional growth inhibition under the various conditions tested was determined as described above. The Bliss expectation (E) for a combined response was calculated by the equation: E=(A+B)−(A×B) where A and B are the fractional growth inhibitions of drug A and B at a given concentration. The difference between the Bliss expectation and the observed growth inhibition of the combination of drugs A and B at the same dose is defined as “Excess over Bliss.” Excess over Bliss scores=0 indicates that the combination treatment is additive (as expected for independent pathway effects); Excess over Bliss scores>0 indicates activity greater than additive (synergy); and Excess over Bliss scores<0 indicates the combination is less than additive (antagonism). Each synergy experiment was done two independent times, in duplicate.

Metabolomics screen and Reverse Phase Protein Array (RPPA). Changes in expression of 301 individual metabolites in LN229 cells treated with PX-866 (10 μM for 48 h) were determined in a global metaholomics screening as described previously. For RPPA, PC3 or LN229 cells were treated with vehicle. PX-866 (10 μM) or the combination of PX-866 plus Gamitrinib (5 μM) for 24 h. Data in triplicate samples were normalized for protein loading, converted to linear values and then log2 transformed.

Bioinformatics analysis. Transcriptional changes in the human kinome following PI3K inhibition were analyzed using OpenArray real-time PCR amplification. GBM organotypic cultures were treated with vehicle (DMSO), PX-866 (10 μM), alone or in combination with Gamitrinib (10 μM) for 48 h. Total RNA was extracted from each sample, reverse-transcribed and the derived cDNA per condition was hybridized with TaqMan® Array Human Protein Kinase Pathways (cat. n. 4414076). Ct values from the experiment were exported from BioTrove OpenArray Real-Time qPCR Analysis Software v1.0.4. ΔΔCt value method was used to calculate fold changes between sample pairs with endogenous control Ct value calculated as average among 15 endogenous controls on the array: ALAS1, B2M, CASC3, G6PD, GAPDH, GUSB, HMBS, HPRT1, IPO8, POLR2A, PPIA, RPLP0, TFRC, UBE2D2, and YWHAZ. P-values for differential expression were calculated using told changes of 15 endogenous controls as a null distribution. FDR values were estimated using Benjamini-Hochberg correction for multiple testing and results with FDR<20% were used for enrichment analysis with Ingenuity Pathway Analysis software (Ingenuity Systems, Richmond, Calif.) to identify a list of pathways and functions significantly overrepresented among the significantly changed genes.

Orthotopic glioblastoma xenograft model. All animal experiments were approved by an Institutional Animal Care and Use Committee. Six-to-eight week old immunodeficient nude mice (Charles River laboratories) were stereotactically injected with 1×10⁵U87-Luc GBM cells expressing luciferease into the right striatum, as described. Established tumors were confirmed by bioluminescence imaging on a Xenogen in Vivo Imaging System after i.p injection of 110 mg/kg D-luciferin, as described previously. Mice with established tumors were assigned to 4 different treatment groups, receiving vehicle (4 animals), Gamitrinib-G4 (2 mg/kg) (4 animals), NVP-BEZ235 25 mg/kg (4 animals) or the combination of both agents (5 animals). Animals were injected i.p. with the various agents, alone or in combination for two rounds with a 5 days on/2 days off schedule, and analyzed weekly by quantitative bioluminescence imaging (photons/s) to monitor differential kinetics of tumor growth in the various groups. The primary endpoint was either a moribund state or death of the animal in accordance with the Institutional Animal Welfare Regulations. At the end of the experiment, tumor samples from the various treatment groups were collected and analyzed histologically for changes in apoptosis or cell proliferation by TUNEL or Ki67 staining, respectively.

Statistical analysis. Data were analyzed using the two-sided unpaired t tests using a GraphPad software package (Prism 4.0) for Windows. Data are expressed as mean±SEM of replicates of a representative experiment out of at least two independent determinations. A p value of ≤0.05 was considered as statistically significant. For RPDA screens, the False Discovery Rate (FDR) values were estimated using Benjamini-Hochberg correction for multiple testing and changes with FDR<20% of at least 1.2 fold were called statistically significant. For metabolomics profiling, missing values (if any) were assumed to be below the level of detection. However, biochemicals that were detected in all samples from one or more groups, but not in samples from other groups, were assumed to be near the lower limit of detection in the groups in which they were not detected. In this case, the lowest detected level of these biochemicals was imputed for samples in which that biochemical was not detected. Following log transformation and imputation with minimum observed values for each compound, Welch's two-sample t-test was used to identify biochemicals that differed significantly between experimental groups. Pathways were assigned for each metabolite, allowing examination of overrepresented pathways.

### Example 2: Results

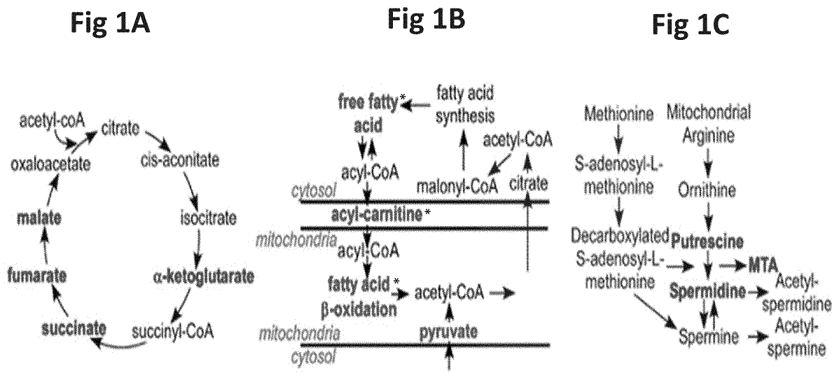

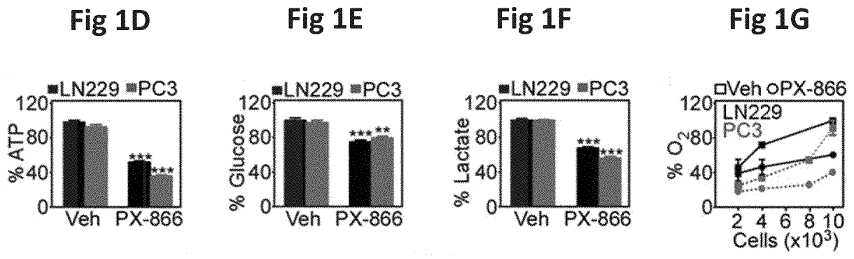

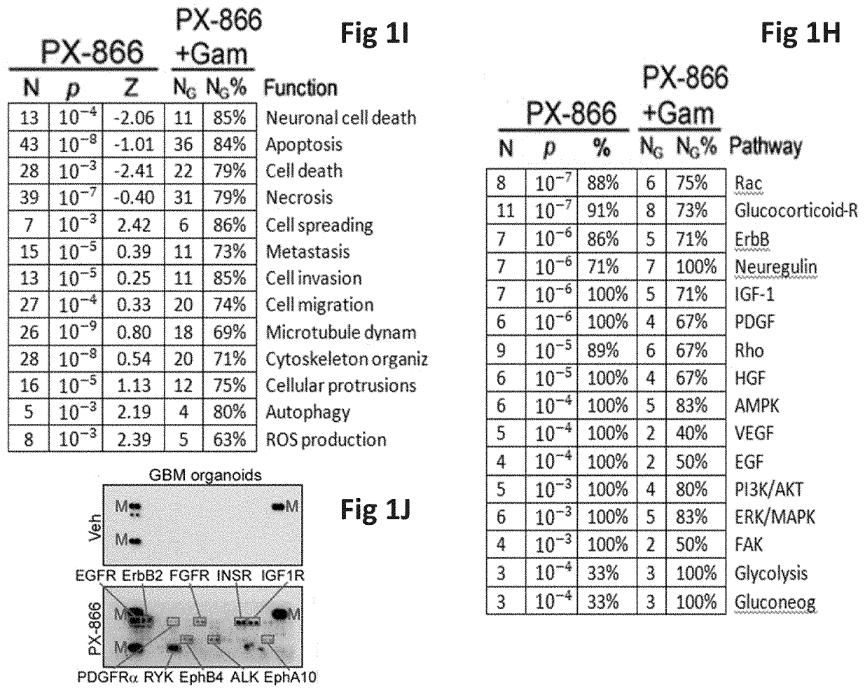

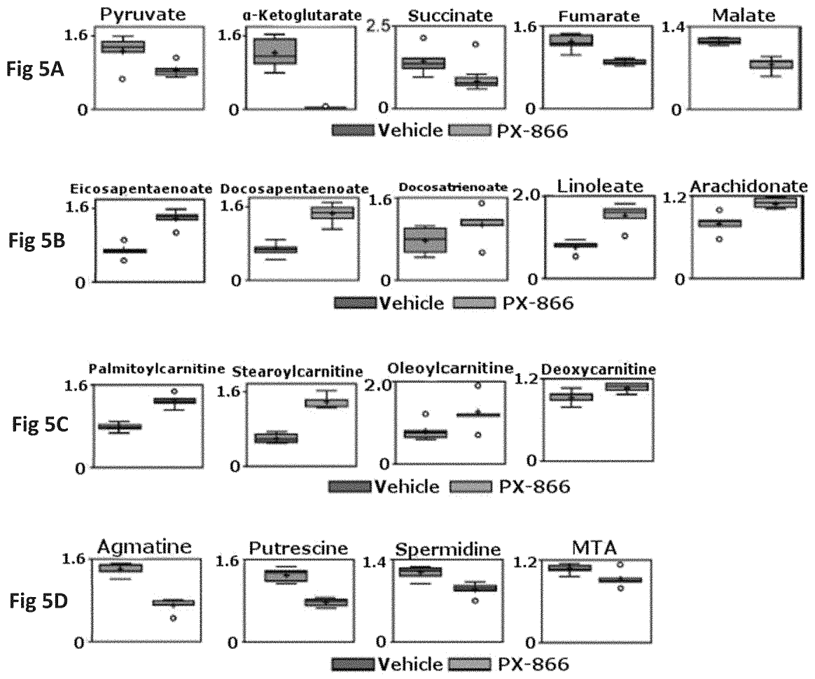

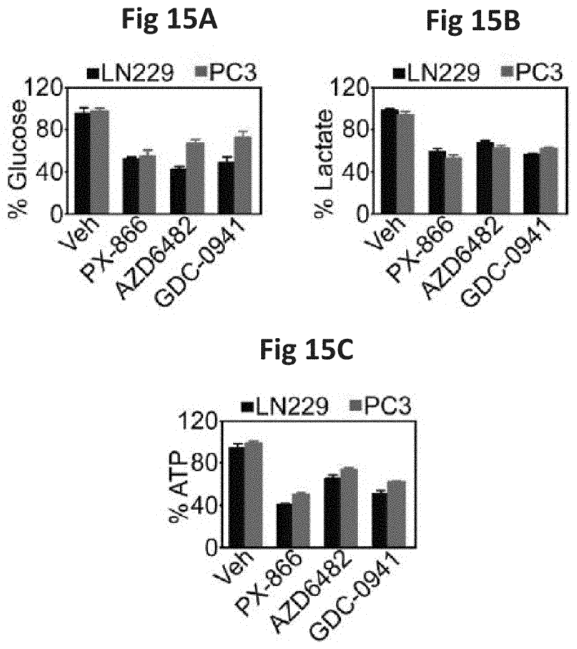

To understand the impact of PI3K inhibition on tumor behavior, we profiled the metaholome of glioblastoma (GBM) LN229 cells exposed to PX-866, a small molecule inhibitor of all PI3K subunits, currently evaluated in clinical trials. PX-866 treatment induced extensive metabolic reprogramming in tumors, especially affecting mitochondrial functions. This involved loss of metabolites involved in oxidative phosphorylation with reduced levels of pyruvate, α-ketoglutarate, succinate, fumarate, and malate (FIG. 1a, FIG. 5a), increased levels of long-chain fatty acids and carnitine conjugates required for mitochondrial fatty acid β-oxidation (FIG. 1b and FIGS. 5b-c), and defective arginine-directed polyamine metabolism, with decreased expression of polyamines, agmatine, spermidine, putrescine, and 5′-deoxy-5′-(methylthio)adenosine (MTA) (FIG. 1c and FIGS. 5c-d). Consistent with these data, LN229 or prostate adenocarcinoma PC3 cells exposed to PI3K inhibitors, including PX-866, AZD6482, or GDC0941 exhibited reduced ATP production (FIG. 1d and FIG. 15c). This resulted from both inhibition of glycolysis with less glucose utilization (FIG. 1e and FIG. 15a) and lactate production (FIG. 1f), and suppression of oxygen consumption (FIG. 1g), a marker of oxidative phosphorylation. As a result of these bioenergetics defects, PI3K therapy considerably reduced adenosine triphosphate (ATP) production in tumor cells (FIG. 15c).

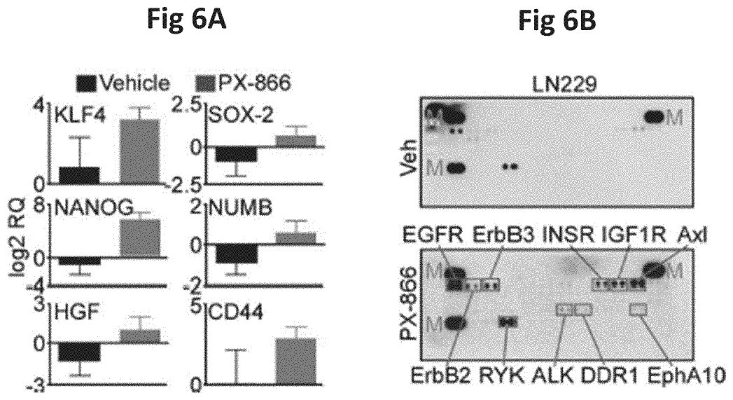

Next, we monitored the tumor kinome in response to PI3K inhibition. In a model of patient-derived and treatment-naive GBM organotypic cultures, we found that PX-866 treatment increased the expression of genes associated with growth factor signaling (EGF, ERK/MAPK, VEGF, ErbB, PDGF), metabolic sensing (AMPK, insulin and glucocorticoid receptors), cytoskeletal remodeling (Rho, Rac), and cell movement (FAK, HGF) (FIG. 1i). These transcriptional changes clustered into two main gene networks of resistance to cell death and increased cell motility (FIG. 1j). In both GBM organoids (FIG. 1J) and LN229 cells (FIG. 6B), this was associated with phosphorylation of several growth factor receptors (EGFR and related members, Insulin-R, IGF1-R, FGFR-2α and PDGFR), as well as kinases (RYK, ALK, DDR1, Axl and Ephrin) implicated in cell movement. Regulators of “sternness”, epithelial-mesenchymal transition, and cell motility (KLF4, NUMB, CD44, NANOG, and HGF) were also transcriptionally modulated by PI3K inhibition (FIG. 6A).

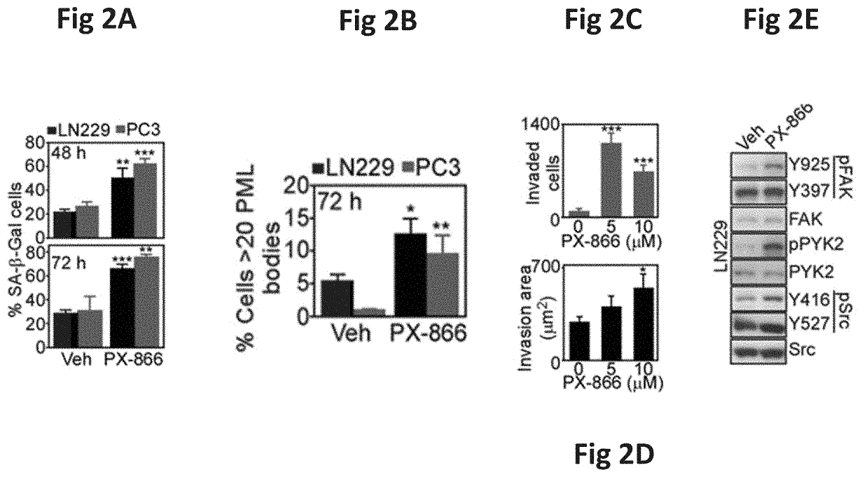

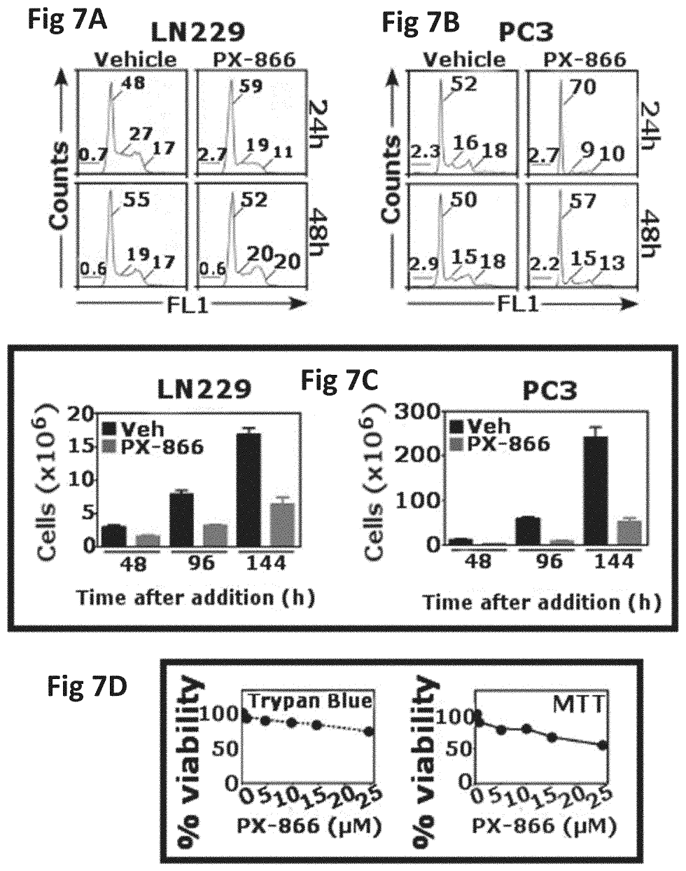

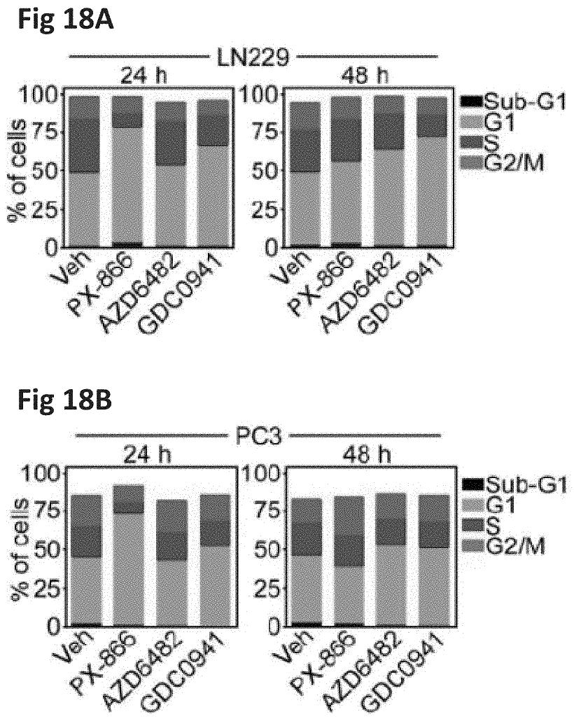

We next asked whether these bioenergetics and transcriptional changes caused a new tumor phenotype. Following treatment with PX-866, tumor cells acquired markers of senescence with increased β-galactosidase staining (SA-β-Gal) (FIG. 2a) and higher PML nuclear body number (FIG. 2b). They also became quiescent, arrested in the G1 phase of the cell cycle (FIGS. 7a,b and FIGS. 18a-b), and had reduced proliferation (FIG. 7c), without significant decrease in viability (FIG. 7d). These cytostatic effects were transient, as the long-term colony forming ability of tumor cells treated with PI3K inhibitors was unaffected compared with control cultures (PC3 or LN229 cells were treated with vehicle (Veh) or PX-866 (10 μM) and analyzed in a colony formation assay after 12 days. Colonies were stained with crystal violet. (Data not shown)).

Instead, PI3K inhibition triggered considerably enhanced tumor cell invasion across Matrigel-coated Transwell membranes (FIG. 2c), as well as in 3D spheroids embedded in a collagen matrix (FIG. 2d). This was associated with higher phosphorylation of several cell motility kinases (FIG. 2e), and this was relevant because silencing of FAK or Src by small interfering RNA reduced tumor cell invasion induced by PI3K inhibition. We also observed that knockdown of Akt isoforms had the same effect, and abolished the increased tumor cell invasion.

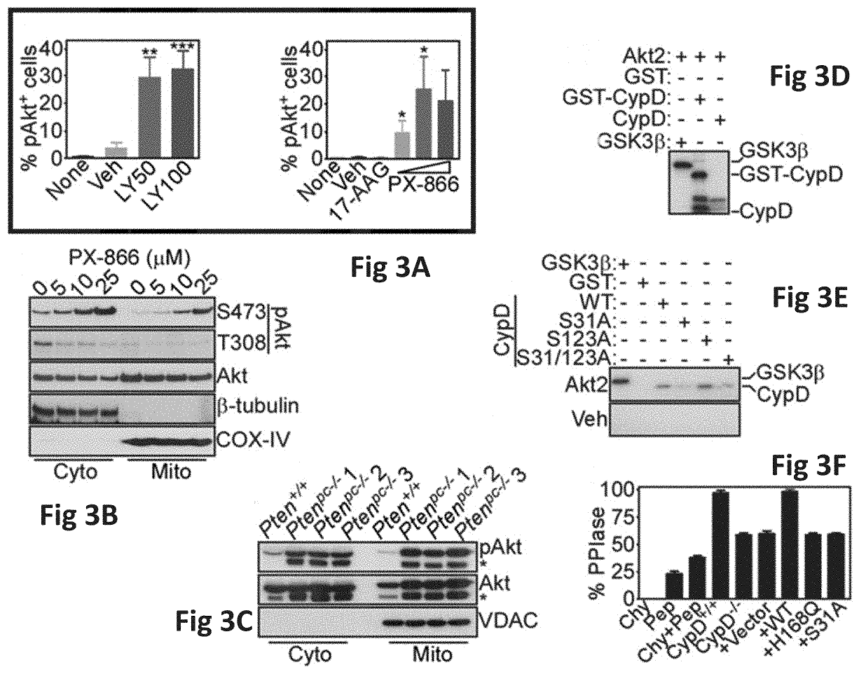

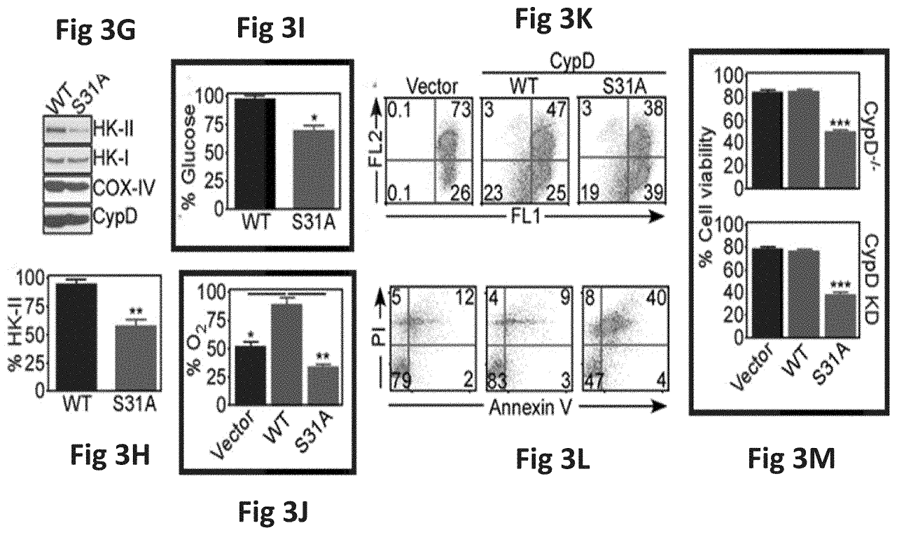

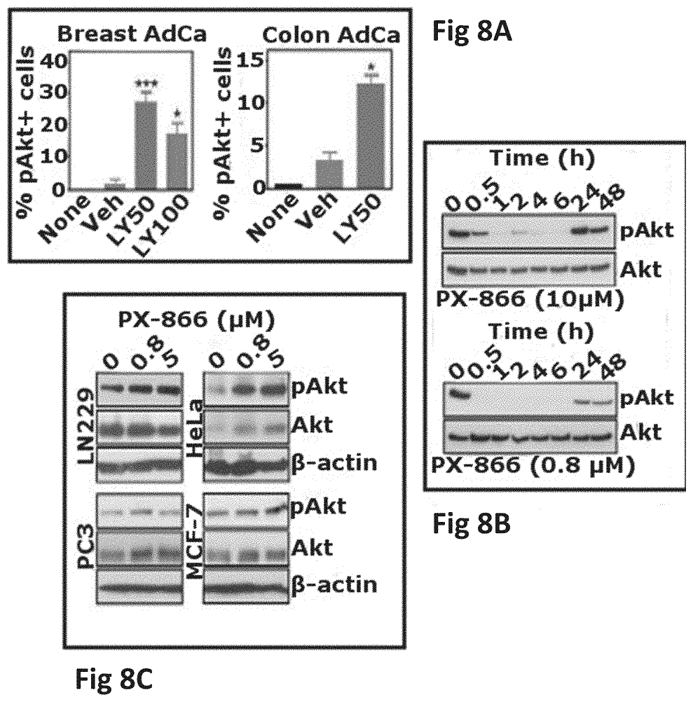

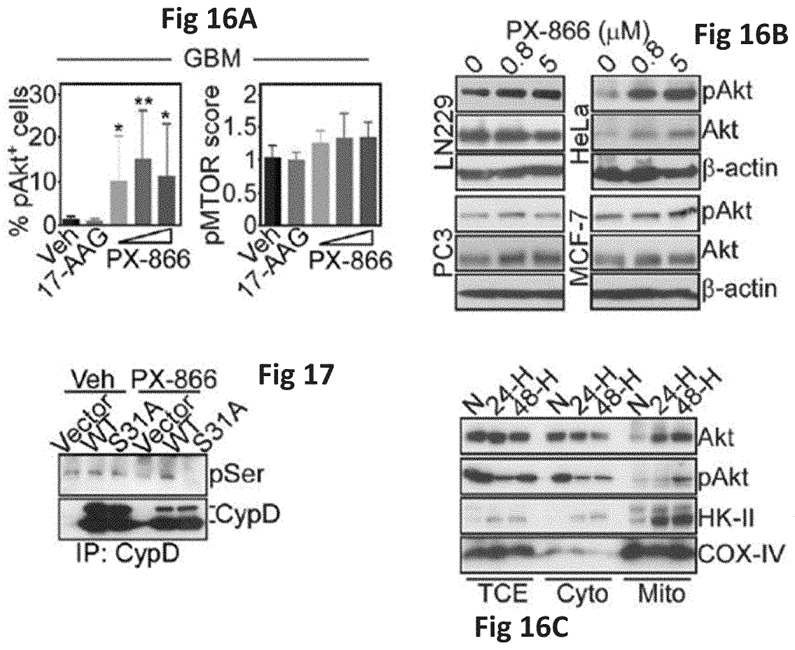

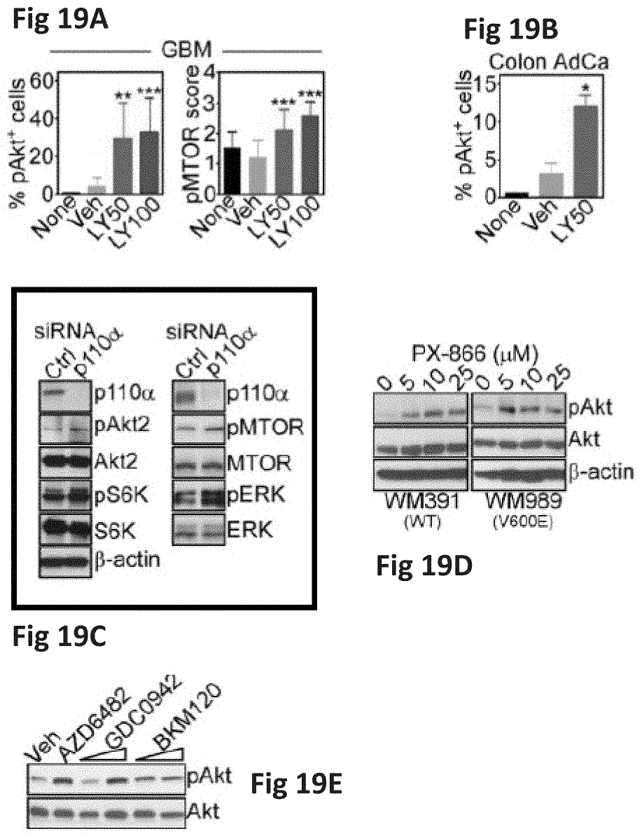

Given these results, we next focused on a potential role of Akt (re)activation in the tumor phenotype induced by PI3K inhibition. Organotypic cultures of representative cases of infiltrating ductal breast adenocarcinoma or colon adenocarcinoma were treated with vehicle (Veh) or the indicated concentrations of pan-PI3K inhibitor, LY294002 (LY) and analyzed for changes in Ser473-phosphorylated Akt (pAkt) after 48 h, by fluorescence microscopy (data not shown). DNA was stained with DAPI. Cytokeratin was an epithelial marker. Organotypic cultures of GBN were treated with vehicle (Vela) or the indicated concentrations (μM) of pan-PI3K inhibitor, LY294002 (LY, 50-100 μM) and analyzed for changes in Ser473-phosphorylated Akt (pAkt) after 48 h, by fluorescence microscopy (data not shown) and quantified. We found that tumor organoids (Table 1) treated with PI3K inhibitors (FIG. 3a) exhibited concentration-dependent phosphorylation of Akt on Ser473 (FIG. 3b; FIG. 8c; FIG. 16a; and FIG. 19a) and higher levels of phosphorylated MTOR (FIG. 19a). PI3K inhibition also induced increased Ser473 phosphorylated Akt in organotypic cultures of breast adenocarcinoma.

Other targeted therapies, including a small molecule inhibitor of Heat Shock Protein-90 (Hsp90) in cytosol, 17-allylamino-demethoxygeldanamycin (17-AAG) had no effect (FIG. 3a). Akt re-phosphorylation occurred 1-2 days after PI3K therapy (FIG. 8b), coinciding with tumor reprogramming (FIGS. 1, 2), and was seen in heterogeneous tumor types (FIG. 8c).

As an alternative experimental approach, we next silenced the expression of PI3K p110α subunit by small interfering RNA (siRNA), and looked at changes in signaling pathways. Similar to the results obtained with pharmacologic inhibition, PI3K knockdown in PC3 cells resulted in increased phosphorylation of Akt2, MTOR and its downstream effector, S6K (FIG. 19c). This response was also associated with increased phosphorylation, ie, activation of ERK1/2 (FIG. 19c), in agreement with previous observations. Consistent with these findings, PI3K therapy-induced. Akt phosphorylation was observed in genetically heterogeneous tumor cell lines (FIG. 16b), regardless of the presence of oncogenic “driver” mutation(s), for instance BRAF V600E melanoma cells (FIG. 19d) and in response to structurally diverse PI3K antagonists currently in the clinic, including AZD6482, GDC0942, and BKM120 (FIG. 19e). Both high (10 μM) and low (0.8 μM) concentrations of PX-866 induced Akt phosphorylation in tumor cells within 24 hours of treatment (FIG. 8b).

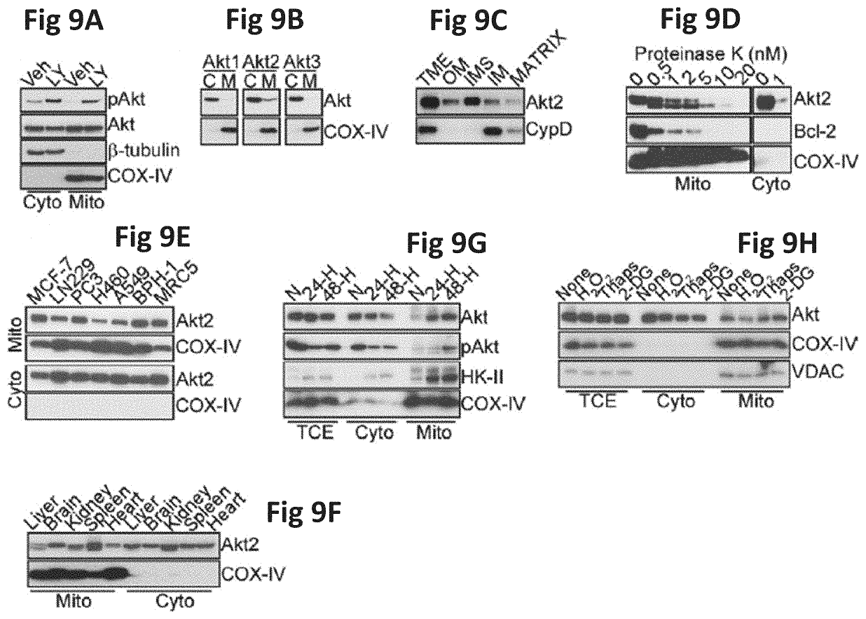

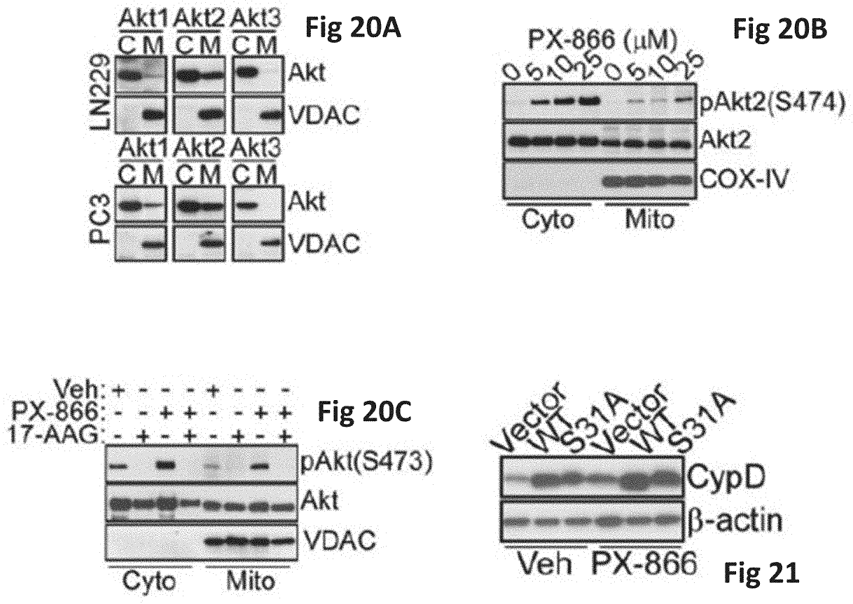

In addition to (re)phosphorylation of Akt in cytosol, PI3K inhibition increased Ser473, phosphorylation of a pool of Akt in mitochondria (FIG. 3b and FIG. 9a). This involved the MTORC2 phosphorylation site on Akt (Ser473), whereas the PDK1 phosphorylation site (Thr308) was unaffected (FIG. 3b). Studying the mitochondrial pool of Akt more in detail, we determined that this was selectively comprised by the Ak2 isoform (FIG. 9b and FIG. 20a), and predominantly localized to the organelle inter-membrane space and inner membrane (FIG. 9c), protected from proteinase K proteolysis of the outer membrane (FIG. 9d). Accordingly, PI3K therapy with PX-866 resulted in robust and concentration-dependent isoform-specific phosphorylation of Akt2 on Ser474 in cytosol and mitochondrial extracts of treated tumor cells (FIG. 20b), as well as primary GBM organotypic cultures from two independent patients (data not shown). Akt is a known client protein for Hsp90, and accordingly pretreatment of tumor cells with 17-AAG abolished the accumulation of phosphorylated Akt in cytosol, as well as mitochondria in response to PX-866 (FIG. 20c). Broadly distributed in tumor cell lines (FIG. 9e) and normal tissues (FIG. 9f) mitochondrial Akt was increased by cellular stress, such as hypoxia (FIG. 9g), or glucose starvation (FIG. 9h), but not oxidative damage (H₂O₂) or endoplasmic reticulum (ER) stress (FIG. 9h).

We next looked for other pathophysiological conditions that may activate mitochondrial Akt, independently of PI3K therapy. Mice with prostate-specific deletion of the Akt inhibitor, Pten, a defect commonly observed in human cancers, also contained high levels of phosphorylated Akt in mitochondria (FIG. 3c). Second, exposure of tumor cells to stress conditions, including hypoxia (FIG. 9d) or glucose starvation induced by the nonmetabolizable analog, 2-deoxyglucose (2-DG), increased Akt recruitment to mitochondria and its phosphorylation on Ser473 (FIG. 9h). In contrast, ER (thapsigargin) or oxidative (H₂O₂) stress had no effect on Akt localization to mitochondria (FIG. 9h).

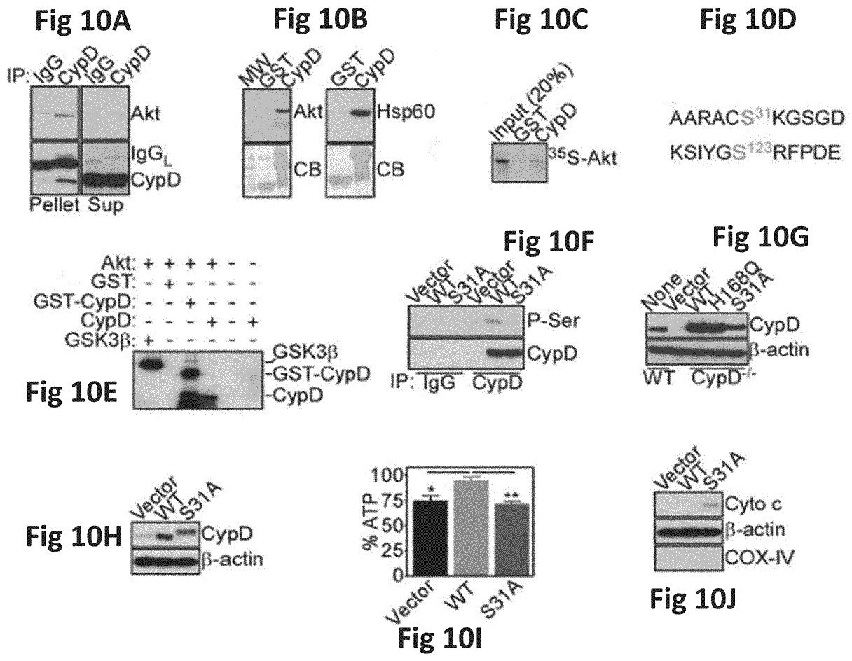

Next, we looked for potential substrate(s) of Akt in mitochondria that could affect tumor reprogramming after PI3K therapy. We found that cyclophilin D (CypD), an important regulator of mitochondrial integrity and bioenergetics contained two potential Akt phosphorylation sites on Ser31, and Ser123 (FIG. 10d). CypD formed a complex with Akt, by immunoprecipitation (FIG. 10a), and pulldown from mitochondrial extracts (FIG. 10b). CypD bound ³⁵S-labeled Akt in a cell-free system (FIG. 10c), suggesting that their interaction was direct. Using kinase assays with recombinant proteins, we found that Akt1 (FIG. 10e) or Akt2 (FIG. 3e) readily phosphorylated wild type (WT) CypD and a CypD Ser123→Ala mutant, Which removes one of the predicted Akt phosphorylation sites in the molecule (FIG. 10d). In contrast, mutagenesis of Ser31→Ala, a second putative phosphorylation site (FIG. 10d), or a CypD Ser31/Ser123→Ala double mutant abolished Akt phosphorylation of CypD (FIG. 3e). When analyzed in vivo, immune complexes containing WT CypD, but not Ser31→Ala mutant, reacted with an antibody to phosphorylated Ser (FIG. 10f), and PX-866 treatment increased this response.

To determine how this phosphorylation event affected mitochondrial functions, we next reconstituted CypD^{-/-} MEFs or LN229 cells with stable short hairpin RNA (shRNA) knockdown of CypD^{≠}with various cDNA constructs (FIG. 10g), Treatment with PX-866 did not affect the levels of endogenous or overexpressed CypD in reconstituted cells (FIG. 21), Under these conditions, WI CypD immunoprecipitated from reconstituted LN229 cells reacted with an antibody to phosphorylated Ser (FIG. 10f). In contrast, immune complexes containing CypD Ser31→Ala mutant did not react with phosphorylated Ser, and immune precipitates with nonbinding IgG were ineffective (FIG. 10f). Similarly, WT CypD immunoprecipitated from reconstituted LN229 cells after treatment with PX-866 showed increased reactivity with an antibody to pSer, compared with control transfectants (FIG. 17). In contrast, pSer reactivity of CypD Ser31→Ala mutant was abolished in PX-866treated cells, and exposure to vehicle did not affect CypD phosphorylation (FIG. 17).

We looked at the function of CypD as a peptidyl prolyl cis, trans isomerase, and found that expression of Akt phosphorylation-defective Ser31→Ala CypD mutant failed to restore this activity in transfected cells, differently from WT CypD (FIG. 3f). As control, reconstitution of CypD−/− MEFs with a PPlase-defective CypD His168→Gln mutant was also ineffective. The isomerase activity of CypD is important to localize the first enzyme of the glycolytic cascade, hexokinase-II (HK-II), to the mitochondrial outer membrane, and this interaction is required for bioenergetics. This response was defective in cells reconstituted with Ser31→Ala CypD mutant (FIG. 10h), with detachment of HK-II from mitochondria (FIG. 3g), and reduction in HK-II activity (FIG. 3h). This caused bioenergetics defects similar to those induced by PI3K inhibition (FIGS. 1d-g), with loss of ATP production (FIG. 10i), and reduced glucose utilization (FIG. 3i) and oxygen consumption (FIG. 3j). CypD is also known for an important role in mitochondrial cell death as a component of the permeability transition pore. Expression of Ser31→Ala CypD mutant was sufficient to promote mitochondrial apoptosis, with dissipation of membrane potential (FIG. 3k), increased Annexin V labeling (FIG. 3l), and release of cytochrome c (FIG. 10j), resulting loss of cell viability in reconstituted cultures (FIG. 3m).

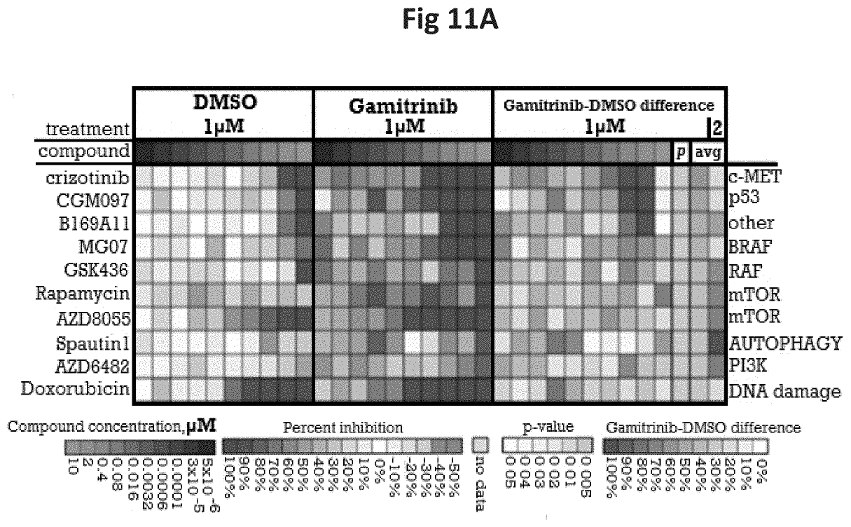

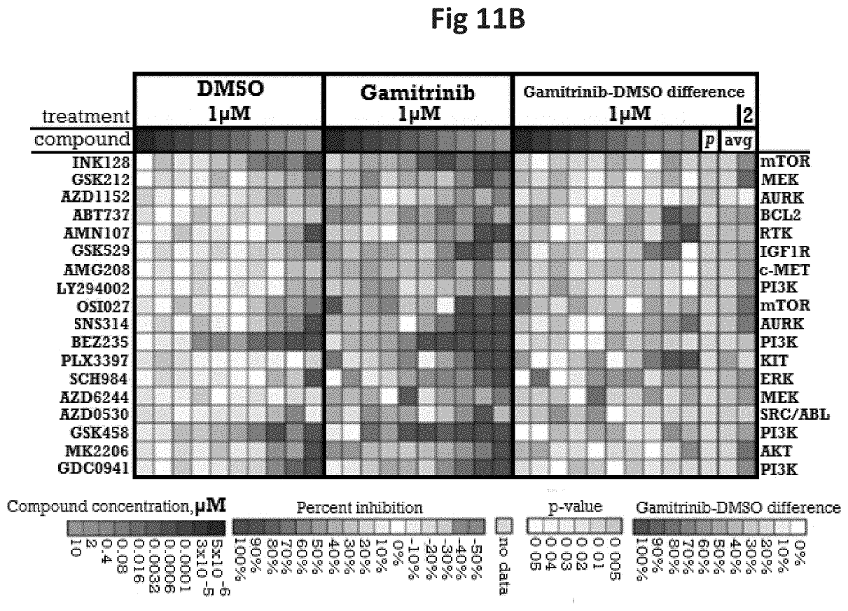

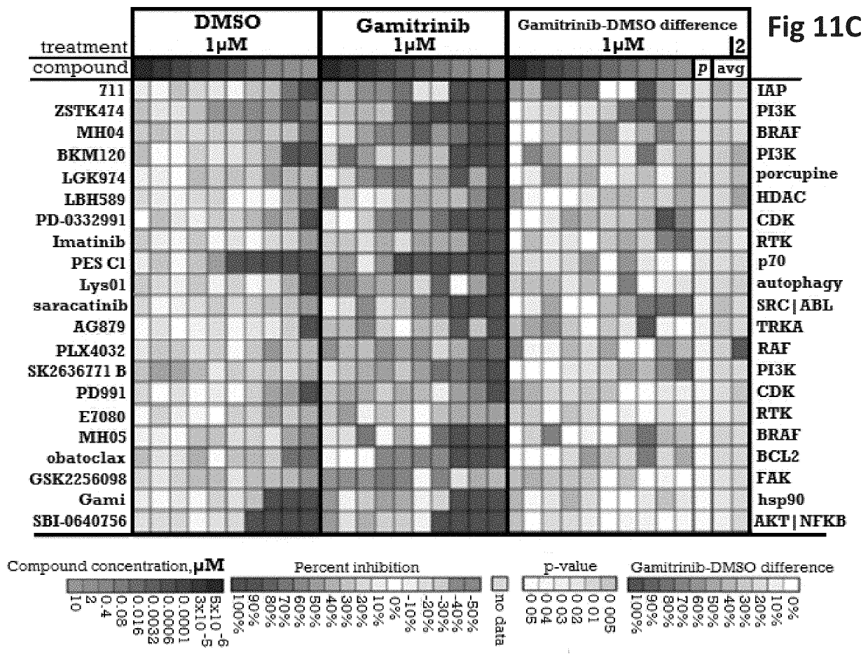

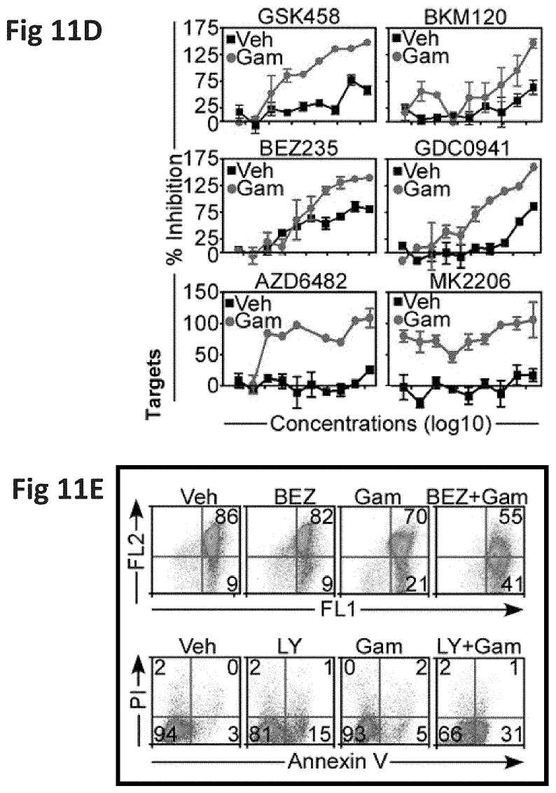

Among the regulators of CypD are mitochondrial-localized chaperones of the Hsp90 family, including Hsp90 and its related homolog, TRAP-1. These molecules maintain organelle protein folding quality control, including of CypD as part of a general adaptive response in cancer. Therefore, we asked whether a chaperone regulation of CypD in mitochondrial reprogramming offered therapeutic opportunities, and we conducted a high-throughput screening combining a selective small molecule inhibitor of mitochondrial Hsp90s, Gamitrinib with various targeted therapies (FIGS. 11A, 11B, and 11C, Table 2 below

TABLE 2
Percent inhibition of increasing concentrations of various compounds in
combination with DMSO (control) or Gamitrinib (Gami).

| conc | | BKM120 | | GSK458 | | BEZ235 | | GDC0941 | | AZD6482 | |
|---|---|---|---|---|---|---|---|---|---|---|---|
| (uM) | log[uM] | DMSO | Gami | DMSO | Gami | DMSO | Gami | DMSO | Gami | DMSO | Gami |
| 10 | 1 | 61.67 | 152.02 | 53.59 | 156.27 | 71.61 | 141.44 | 64.84 | 156.27 | 43.43 | 141.10 |
| 2 | 0.30103 | 54.89 | 151.13 | 63.10 | 142.85 | 67.61 | 137.11 | 58.94 | 140.39 | 5.91 | 133.90 |
| 0.4 | −0.39794 | 50.36 | 150.75 | 58.11 | 138.84 | 65.07 | 140.20 | 29.77 | 134.46 | −7.43 | 114.08 |
| 0.08 | −1.09691 | 17.73 | 153.91 | 67.49 | 133.47 | 62.57 | 133.41 | 21.25 | 121.46 | 21.68 | 111.71 |
| 0.016 | −1.79588 | 8.78 | 138.62 | 52.35 | 136.49 | 74.60 | 145.76 | −0.14 | 126.85 | 10.82 | 118.13 |
| 0.0032 | −2.49485 | 3.42 | 140.77 | 38.13 | 139.92 | 60.70 | 134.66 | −17.33 | 19.20 | −4.10 | 123.40 |
| 0.00064 | −3.19382 | 16.22 | 130.27 | 11.32 | 139.64 | 23.82 | 127.29 | 7.03 | 133.55 | −10.50 | 128.16 |
| 0.000128 | −3.89279 | 4.38 | 132.78 | 8.89 | 135.48 | −21.36 | 122.82 | −14.66 | 124.43 | 11.82 | 123.03 |
| 2.56E−05 | −4.59176 | 19.80 | 136.65 | 1.66 | 128.82 | −9.35 | 121.84 | −27.15 | 104.45 | −2.03 | 119.70 |
| 5.12E−06 | −5.29073 | 14.64 | 130.32 | 17.03 | 132.69 | −7.25 | 119.42 | 0.41 | 125.94 | 3.23 | 129.84 |

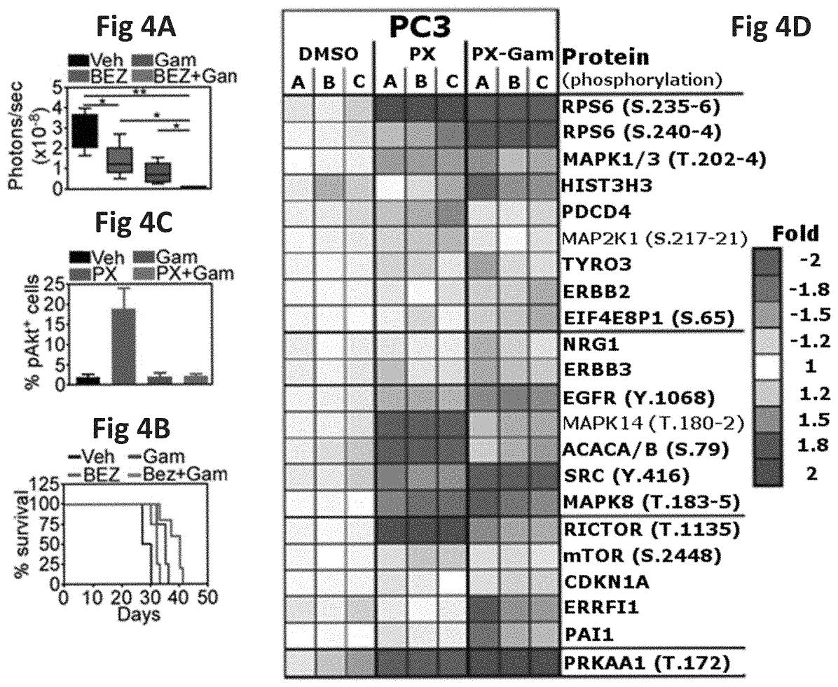

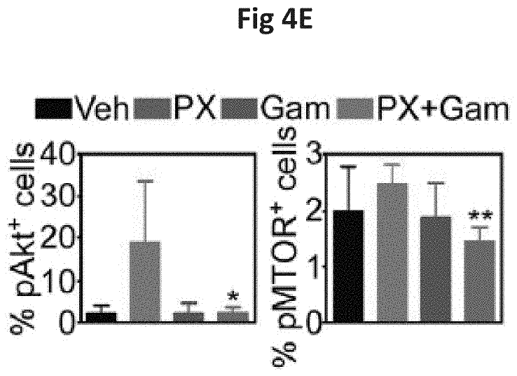

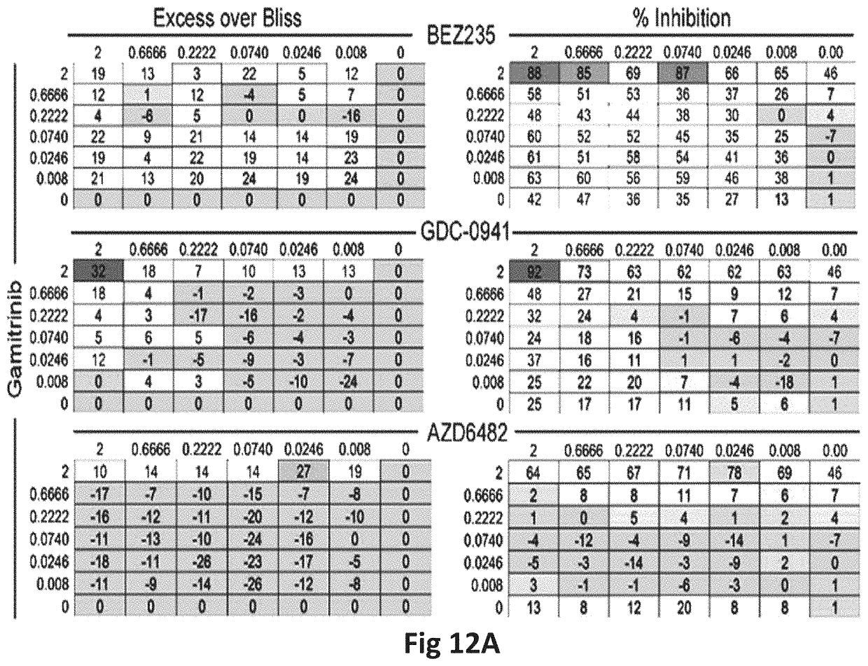

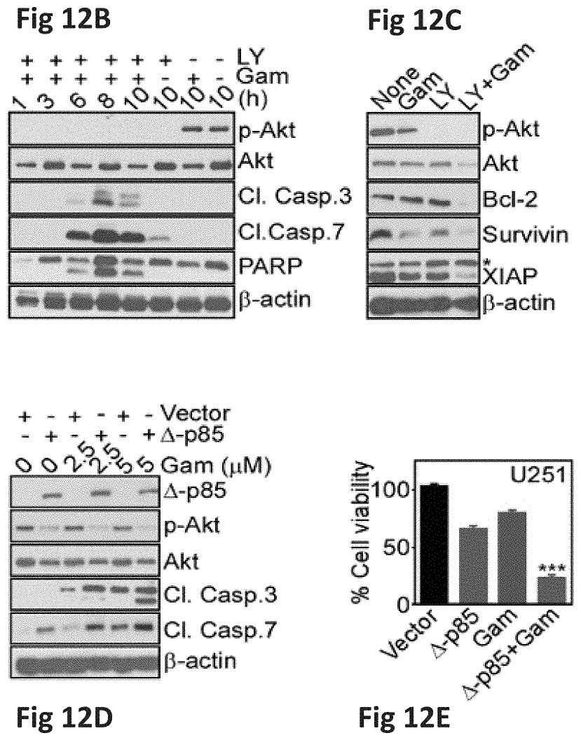

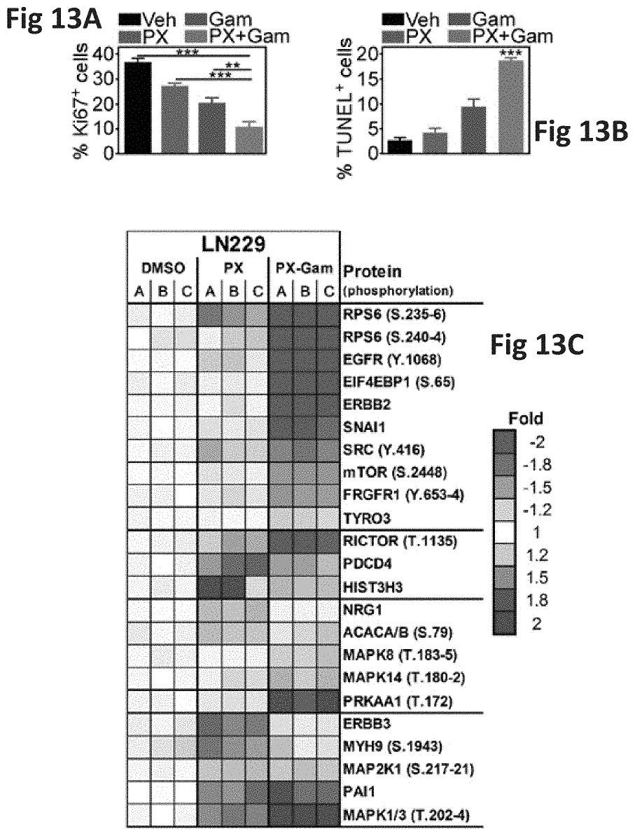

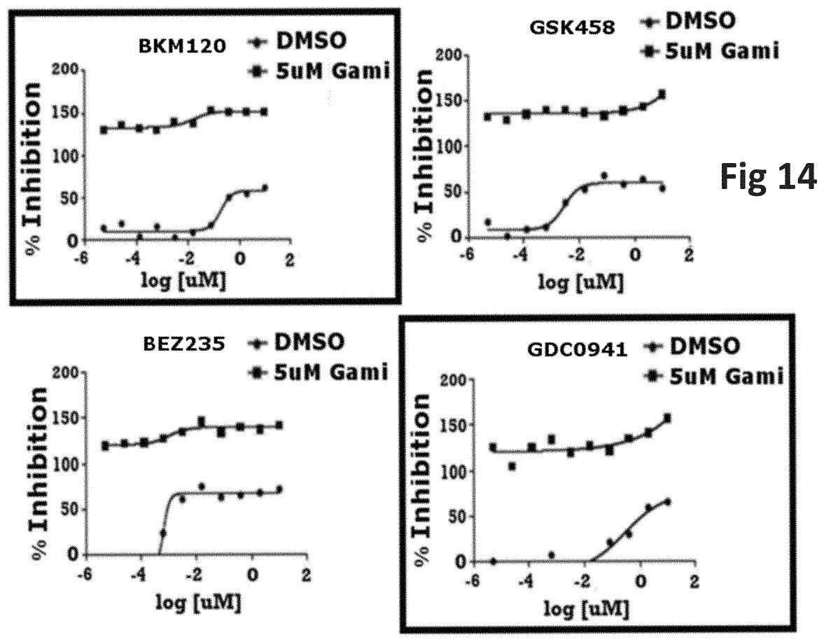

We found that Gamitrinib potently enhanced the anticancer activity of PI3K/Akt/MTOR pathway antagonists (FIG. 11D, FIG. 14, Table 2). This effect was specific, as other targeted therapies were unaffected by Gamitrinib (FIGS. 11A, 11B, and 11C), and synergistically inhibited tumor cell proliferation, by Bliss independence analysis (FIG. 12a). The combination of Gamitrinib plus PI3K inhibitors caused extensive mitochondrial apoptosis¹⁵, with loss of membrane potential (FIG. 11E (top)), Annexin V labeling (FIG. 11E (bottom)), and cleavage of effector caspase-3 and -7, and their substrate, poly-ADP ribose polymerase (PARD) (FIG. 12b). Several anti-apoptotic molecules exploited for tumor cell survival, including Bcl-2, XIAP and survivin, were degraded under these conditions (FIG. 12c). Targeting the PI3K pathway with transfection of a dominant negative Δ-p85 subunit that interferes with PI3K signaling instead of small molecule inhibitors also synergized with Gamitrinib in producing caspase cleavage (FIG. 12d), and enhanced tumor cell killing (FIG. 12e). When tested in an orthotopic model of intracranial GBM in mice, the combination of Gamitrinib plus a PI3K antagonist (NVP-BEZ235) inhibited glioma growth (FIG. 4a), and extended animal survival, compared to each treatment alone (FIG. 4b). Nude mice were injected with U87-Luc cells in the right cerebral striatum, treated with vehicle (Veh), NVP-BEZ235 (BEZ), or Gamitrinib (Gam) alone or in combination, and analyzed for luciferase expression by bioluminescence imaging at the indicated time intervals after injection (photos not shown). Histologically, these tumors showed reduced tumor cell proliferation (FIG. 13a), and increased apoptosis (FIG. 13b), in vivo, compared with tumors in groups receiving each agent alone.

In addition to CypD, the mitochondrial Hsp90 proteome comprises multiple regulators of organelle function⁷, and we asked whether tumor reprogramming induced by PI3K inhibition required global organelle integrity. Organotypic cultures of a representative case of infiltrating ductal breast adenocarcinoma treated with vehicle (Veh) or Gamitrinib (5 μM) were analyzed by immunohistochemistry and fluorescence microscopy after 48 h. HE, hematoxylin & eosin staining. Cytokeratin (CK) was used as an epithelial marker. We found that addition of Gamitrinib to tumor organoids largely reversed the transcriptional changes induced by PI3K inhibition (FIGS. 1h,i), and prevented the reactivation of Akt in these settings (data not shown) and no longer promoted the phosphorylation of MTOR (FIG. 4e). When analyzed in a reverse phase protein array, the combination of Gamitrinib plus PX-866 reversed many of the signaling pathways activated by PI3K inhibition, including Akt/MTOR (4EBP1, S6, MTOR, RICTOR), and EGF (EGFR, ErbB2, ErbB3, NRG, ERRFI) responses, blocked changes in cell invasion and metastasis effectors (Snail, Tyro3, Src, PAI1), promoted cell cycle arrest (CDKN1A, MAPK8, MAPK14), and activated metabolic tumor suppression (PRKAA1) (FIG. 4d and FIG. 13c).

Therefore, molecular therapy functions as a “stress” driver of new cancer phenotypes, repurposing mitochondrial functions to promote tumor selection. The combined phenotype induced by PI3K inhibition of reversible senescence, transient quiescence, heightened cell survival and increased invasion is poised to facilitate early tumor cell dissemination and metastatic seeding at distant sites, two requirements of tumor dormancy and treatment resistance. Disabling mitochondrial reprogramming can reverse these traits and provide a broad approach to enhance anticancer activity irrespective of tumor heterogeneity.

### Example 3: Discussion

In this study, we have shown that PI3K therapy currently in the clinic is a powerful driver of tumor adaptation, reprogramming mitochondrial functions in bioenergetics and apoptosis to promote cell survival and treatment resistance. This pathway is centered on a pool of Akt2 recruited to mitochondria, and its phosphorylation of the mitochondrial regulator, CypD, on Ser31. Conversely, the combination of PI3K therapy with an antagonist of CypD protein folding currently in preclinical development, Gamitrinib, reverses this adaptive response and delivers potent, synergistic anticancer activity in vivo.

Despite their ability to target a fundamental cancer node, small-molecule inhibitors of PI3K/Akt/MTOR have shown modest efficacy in the clinic. The data presented here identify the paradoxical reactivation of Akt in response to PI3K therapy, as a pivotal effector of drug resistance to these regimens. Centered on the recruitment of Akt2 to mitochondria, this pathway differs from other mechanisms of drug resistance mediated by intratumor heterogeneity, acquisition of new mutations, or crosstalk within the tumor microenvironment.

Once in mitochondria, Akt2 associated with the organelle regulator CypD and phosphorylated CypD on Ser31 to preserve its PPIase activity, maintain energy production, and antagonize apoptosis. There is prior evidence that post-translational modifications, for instance acetylation, affect CypD activity. Here, Ser31 is positioned at the NH2 terminus of the mature form of CypD and becomes readily phosphorylated by Akt in vitro and in vivo. However, the complete Akt consensus phosphorylation site for Ser31 extends into the mitochondrial-import sequence, and it is possible that a fraction of CypD is phosphorylated on Ser31 during mitochondrial trafficking. Akt plays a central role in tumor bioenergetics, influencing aerobic glycolysis, as well as oxidative phosphorylation. An antiapoptotic role of Akt2 phosphorylation of CypD is also consistent with a physical assembly of CypD in a mitochondrial permeability transition pore that regulates stress-associated cell death.

The functions of CypD in bioenergetics and apoptosis require protein folding quality control maintained by mitochondrial-localized Hsp90s. Accordingly, the combination of a small-molecule inhibitor of mitochondrial-localized Hsp90s currently in preclinical development, Gamitrinib, converted a transient, cytostatic effect of PI3K antagonists into potent, synergistic anticancer activity in vivo. The idea of targeting mitochondrial integrity for cancer therapy has recently gained attention, and regulators of Bcl-2 proteins, oxidative phosphorylation, and redox mechanisms have progressed through (pre)clinical development. Gamitrinib is an attractive candidate for this approach given its ability to simultaneously disable multiple pathways of mitochondrial homeostasis in bioenergetics, gene expression, and redox balance selectively in tumors.

In addition, the combination with Gamitrinib reversed adaptive tumor reprogramming induced by PI3K therapy, with respect to Akt (re)activation, growth factor receptor signaling, cell proliferation, and endogenous tumor suppression. Small molecule inhibitors of PI3K, Akt, or MTOR have been shown to activate a broad gene expression program in tumor cells, potentially as a compensatory response via derepression of FOXO-dependent transcription. Our RPPA screening suggests that mitochondrial reprogramming maintained by organelle Hsp90s is important for this response, potentially via mitochondria-to-nuclei “retrograde” signaling. Accordingly, mitochondria-derived “retrograde” mediators that affect nuclear gene expression have been identified in model systems, and CypD contributes to retrograde signaling via activation of STAT3-dependent cell migration and invasion.

All patents, patent applications and other references cited in this specification are hereby incorporated by reference in their entirety, including those listed in the references section below. Specifically, Ghosh et al, Adaptive mitochondrial reprogramming and resistance to PI3K therapy, J. National Cancer Inst, 107(3) (February 2015) is incorporated herein by reference in its entirety.

### REFERENCES

1 Gillies, R. J., Verduzco, D. & Gatenby, R. A. Evolutionary dynamics of carcinogenesis and why targeted therapy does not work. Nat Rev Cancer 12, 487-493 (2012).

2 Fodale, V., Pierobon, M., Liotta, L. & Petricoin, E. Mechanism of cell adaptation: when and how do cancer cells develop chemoresistance? Cancer 117, 89-95 (2011).

3 Goss, P. E. & Chambers, A. F. Does tumour dormancy offer a therapeutic target? Nat Rev Cancer 10, 871-877 (2010).

4 Friedl, P. & Alexander, S. Cancer invasion and the microenvironment: plasticity and reciprocity. Cell 147, 992-1009 (2011).

5 Manning, B. D. & Cantley, L. C. AKT/PKB signaling: navigating downstream. Cell 129, 1261-1274 (2007).

6 Rodon, J., Dienstmann, R., Serra, V. & Tabemero, J. Development of PI3K inhibitors: lessons learned from early clinical trials. Nat Rev Clin Oncol 10, 143-153 (2013).

7 Chae, Y. C. et al. Landscape of the mitochondrial Hsp90 metabolome in tumours. Nat Commun 4, 2139 (2013).

8 Vander Heiden, M. G., Cantley, L. C. & Thompson, C. B. Understanding the Warburg effect: the metabolic requirements of cell proliferation. Science 324, 1029-1033 (2009).

9 Wallace, D. C. Mitochondria and cancer. Nat Rev Cancer 12, 685-698 (2012).

10 Vaira, V. et al. Preclinical model of organotypic culture for pharmacodynamic profiling of human tumors. Proc Natl Acad Sci USA 107, 8352-8356 (2010).

11 Chakrabarty, A., Sanchez, V., Kuha, M. G., Rinehart, C. & Arteaga, C. L. Feedback upregulation of HERS (ErbB3) expression and activity attenuates antitumor effect of PI3K inhibitors. Proc Natl Acad. Sci USA 109, 2718-2723 (2012).

12 De Los Angeles, A., Loh, Y. H., Tesar, P. J. & Daley, G. Q. Accessing naive human pluripotency. Curr Opin Genet Dev 22, 272-282 (2012).

13 Chandarlapaty, S. et al. AKT inhibition relieves feedback suppression of receptor tyrosine kinase expression and activity. Cancer Cell 19, 58-71 (2011).

14 Song, M. S., Salmena, L. & Pandolfi, P. P. The functions and regulation of the PTEN tumour suppressor. Nat Rev Mol Cell Biol 13, 283-296 (2012).

15 Tait. S. W. & Green. D. R. Mitochondria and cell death: outer membrane permeabilization and beyond. Nat Rev Mol Cell Biol 11, 621-632 (2010).

16 Ghosh, J. C., Siegelin, M. D., Dohi, T. & Alfieri, D. C. Heat shock protein 60 regulation of the mitochondrial permeability transition pore in tumor cells. Cancer Res 70, 8988-8993 (2010).

17 Aguirre Ghiso, J. A. Inhibition of FAK signaling activated by urokinase receptor induces dormancy in human carcinoma cells in vivo. Oncogene 21, 2513-2524 (2002).

18 Kang, B. H. et al. Regulation of tumor cell mitochondrial homeostasis by an organelle-specific Hsp90 chaperone network. Cell 131, 257-270 (2007).

19 Taipale, M., Jarosz, D. F. & Lindquist, S. HSP90 at the hub of protein homeostasis: emerging mechanistic insights. Nat Rev Mol Cell Biol 11, 515-528 (2010).

20 Chae, Y. C. et al. Control of tumor bioenergetics and survival stress signaling by mitochondrial HSP90s. Cancer Cell 22, 331-344 (2012).

21 Ebos, J. M. et al. Accelerated metastasis after short-term treatment with a potent inhibitor of tumor angiogenesis. Cancer Cell 15, 232-239 (2009).

22 Greaves, M. & Maley, C. C. Clonal evolution in cancer. Nature 481, 306-313 (2012).

23 Beausejour, C. M. et al. Reversal of human cellular senescence: roles of the p53 and 16 pathways. EMBO J 22, 4212-4222 (2003).

24 Husemann, Y. et al. Systemic spread is an early step in breast cancer. Cancer Cell 13, 58-68 (2008).

25 Buchakjian, M. R. & Kombluth, S. The engine driving the ship: metabolic steering of cell proliferation and death. Nat Rev Mol Cell Biol 11, 715-727 (2010).

26 Vaira, V. et at. Preclinical model of organotypic culture for pharmacodynamic profiling of human tumors. Proc Natl Acad Set USA 107, 8352-8356 (2010).

27 Chae, Y. C. et al. Control of tumor bioenergetics and survival stress signaling by mitochondrial HSP90s. Cancer Cell 22, 331-344 (2012).

28 Ghosh, J. C., Siegelin, M. D., Dohi, T. & Alfieri, D. C. Heat shock protein 60 regulation of the mitochondrial permeability transition pore in tumor cells. Cancer Res 70, 8988-8993 (2010).

29 Schinzel, A. C. et al. Cyclophilin D is a component of mitochondrial permeability transition and mediates neuronal cell death after focal cerebral ischemia. Proc. Natl. Acad. Sci. U S A. 102, 12005-12010 (2005).

30 Kang, B. H. et al. Combinatorial drug design targeting multiple cancer signaling networks controlled by mitochondrial Hsp90. J Clin Invest 119, 454-464 (2009).

31 Kang, B. H. et al. Regulation of tumor cell mitochondrial homeostasis by an organelle-specific Hsp90 chaperone network. Cell 131, 257-270 (2007).

32 Lin, D. T. & Lechleiter, J. D. Mitochondrial targeted cyclophilin D protects cells from cell death by peptidyl prolyl isomerization. J Biol Chem 277, 31134-31141 (2002).

33 O'Connell, M. P. et at. Hypoxia Induces Phenotypic Plasticity and Therapy Resistance in Melanoma via the Tyrosine Kinase Receptors ROR1 and ROR2. Cancer Discov 3, 1378-1393 (2013).

34 Dohi, T., Xia, F. & Altieri, D. C. Compartmentalized phosphorylation of IAP by protein kinase A regulates cytoprotection. Mol Cell 27, 17-28 (2007).

35 Fitzgerald, J. B., Schoeberl, B., Nielsen, U. B. & Sorger, P. K. Systems biology and combination therapy in the quest for clinical efficacy. Nat Chem Biol 2, 458-466 (2006).

36 Chae, Y. C. et al. Landscape of the mitochondrial Hsp90 metabolome in tumours. Nat Commun 4, 2139 (2013).

37 Siegelin, M. D. et al. Exploiting the mitochondrial unfolded protein response for cancer therapy in mice and human cells. J Clin Invest 121, 1349-1360 (2011).

1. A method of treating cancer in a subject in need thereof, comprising administering to the subject a pharmaceutically effective amount of a pharmaceutical composition comprising gamitrinib and a PI3K inhibitor.

2. The method according to claim 1, wherein the PI3K inhibitor is selected from PX-866, AZD6482, LY294002, BEZ235, GSK458, GDC0941, ZSTK474, BKM120 and GSK2636771B.

3. The method according to claim 1, wherein the composition further comprises a pharmaceutically acceptable carrier.

4. The method according to claim 1, wherein the gamitrinib is present in an amount from 1 μg to 100 mg.

5. The method according to claim 1, wherein the PI3K inhibitor is present in an amount from 0.5 mg/kg to about 40 mg/kg of the subject's body weight.

6. The method of claim 15, wherein the subject has cancer.

7. The method of claim 6, wherein the subject has a cancer which is drug resistant.

8. The method of claim 1, wherein the subject has glioblastoma.

9. The method of claim 1, wherein the subject has adenocarcinoma.

10. The method according to claim 5, wherein the PI3K inhibitor is present in an amount of about 1 mg/kg to about 20 mg/kg of the subject's body weight.

11. The method according to claim 1, wherein the PI3K inhibitor is present in an amount of about 100 mg.


SEQUENCE LISTING (2 sequences)

SEQ ID NO: 1 - DNA, 33 bp, Artificial Sequence
  Mutated sequence
gcggcccgcgcctgcgccaagggctccggcgac

SEQ ID NO: 2 - DNA, 41 bp, Artificial Sequence
  Mutated sequence
cgggaagtccatctacggagcccgctttcctgacgagaact